(19) United States
(12) Patent Application Publication
(10) Pub. No.: US 2013/0225463 A1
(43) Pub. Date: Aug. 29, 2013

(54) QUATERNIZED POLYETHER AMINES AND THEIR USE AS ADDITIVE FOR FUELS AND LUBRICANTS

(71) Applicants: Markus HANSCH, Speyer (DE); Harald BOEHNKE, Mannheim (DE); Ludwig VOELKEL, Limburgerhof (DE); Marc WALTER, Frankenthal (DE); Wolfgang GRABARSE, Mannheim (DE)

(72) Inventors: Markus HANSCH, Speyer (DE); Harald BOEHNKE, Mannheim (DE); Ludwig VOELKEL, Limburgerhof (DE); Marc WALTER, Frankenthal (DE); Wolfgang GRABARSE, Mannheim (DE)

(21) Appl. No.: 13/668,985

(22) Filed: Nov. 5, 2012

Related U.S. Application Data
(60) Provisional application No. 61/555,573, filed on Nov. 4, 2011.

(51) Int. Cl. C10L 1/222; C07C 213/02; C10M 133/06; C07C 217/08

(52) U.S. Cl. CPC C10L 1/2225; C07C 217/08; C07C 213/02; C10M 133/06
USPC 508/547; 44/434; 564/292

(57) ABSTRACT

The present invention relates to novel quaternized polyetheramines and to the preparation thereof. The present invention further relates to the use of these compounds as a fuel and lubricant additive. More particularly, the invention relates to the use of these quaternized nitrogen compounds as a fuel additive for reducing or preventing deposits in the injection systems of direct injection diesel engines, especially in common rail injection systems, for reducing the fuel consumption of direct injection diesel engines, especially of diesel engines with common rail injection systems, and for minimizing power loss in direct injection diesel engines, especially in diesel engines with common rail injection systems. The invention also provides additive packages comprising these polyetheramines; and fuels and lubricants additized therewith. The invention further relates to the use of these quaternized nitrogen compounds as an additive for gasoline fuels, especially for improving the intake system cleanliness of gasoline engines.

The present invention relates to novel quaternized polyetheramines and to the preparation thereof. The present invention further relates to the use of these compounds as a fuel and lubricant additive. More particularly, the invention relates to the use of these quaternized nitrogen compounds as a fuel additive for reducing or preventing deposits in the injection systems of direct injection diesel engines, especially in common rail injection systems, for reducing the fuel consumption of direct injection diesel engines, especially of diesel engines with common rail injection systems, and for minimizing power loss in direct injection diesel engines, especially in diesel engines with common rail injection systems. The invention also provides additive packages comprising these polyetheramines; and fuels and lubricants additized therewith. The invention further relates to the use of these quaternized nitrogen compounds as an additive for gasoline fuels, especially for improving the intake system cleanliness of gasoline engines.

### STATE OF THE ART

In direct injection diesel engines, the fuel is injected and distributed ultrafinely (nebulized) by a multihole injection nozzle which reaches directly into the combustion chamber in the engine, instead of being introduced into a prechamber or swirl chamber as in the case of the conventional (chamber) diesel engine. The advantage of the direct injection diesel engines lies in their high performance for diesel engines and nevertheless low fuel consumption. Moreover, these engines achieve a very high torque even at low speeds.

At present, essentially three methods are being used to inject the fuel directly into the combustion chamber of the diesel engine: the conventional distributor injection pump, the pump-nozzle system (unit-injector system or unit-pump system) and the common rail system.

In the common rail system, the diesel fuel is conveyed by a pump with pressures up to 2000 bar into a high-pressure line, the common rail. Proceeding from the common rail, branch lines run to the different injectors which inject the fuel directly into the combustion chamber. The full pressure is always applied to the common rail, which enables multiple injection or a specific injection form. In the other injection systems, in contrast, only smaller variation in the injection is possible. The injection in the common rail is divided essentially into three groups: (1.) pre-injection, by which essentially softer combustion is achieved, such that harsh combustion noises (“nailing”) are reduced and the engine seems to run quietly; (2.) main injection, which is responsible especially for a good torque profile; and (3.) post-injection, which especially ensures a low NOₓ value. In this post-injection, the fuel is generally not combusted, but instead evaporated by residual heat in the cylinder. The exhaust gas/fuel mixture formed is transported to the exhaust gas system, where the fuel, in the presence of suitable catalysts, acts as a reducing agent for the nitrogen oxides NOₓ.

The variable, cylinder-individual injection in the common rail injection system can positively influence the pollutant emission of the engine, for example the emission of nitrogen oxides (NO_{X}), carbon monoxide (CO) and especially of particulates (soot). This makes it possible, for example, for engines equipped with common rail injection systems can meet the Euro 4 standard theoretically even without additional particulate filters.

In modern common rail diesel engines, under particular conditions, for example when biodiesel-containing fuels or fuels with metal impurities such as zinc compounds, copper compounds, lead compounds and other metal compounds are used, deposits can form on the injector orifices, which adversely affect the injection performance of the fuel and hence impair the performance of the engine, i.e. especially reduce the power, but in some cases also worsen the combustion. The formation of deposits is enhanced further by further developments in the injector construction, especially by the change in the geometry of the nozzles (narrower, conical orifices with rounded outlet). For lasting optimal functioning of engine and injectors, such deposits in the nozzle orifices must be prevented or reduced by suitable fuel additives.

Carburetors and intake systems of gasoline engines, but also injectors of injection systems for fuel dosage, are contaminated by impurities which are caused by dust particles from the air, uncombusted hydrocarbon residues from the combustion chamber and the crankcase ventilation gases passed into the carburetor.

These residues shift the air-fuel ratio when idling and in the lower partial load range, such that the mixture becomes leaner, the combustion becomes less complete and, in turn, the proportions of uncombusted or partly combusted hydrocarbons in the exhaust gas become greater and the gasoline consumption rises.

It is known that these disadvantages are avoided by using fuel additives to maintain cleanliness of valves and carburetors or injection systems of gasoline engines (cf., for example: M. Rossenbeck in Katalysatoren, Tenside, Mineralöladditive [Catalysts, Surfactants, Mineral Oil Additives], eds. J. Falbe, U. Hasserodt, p. 223, G. Thieme Verlag, Stuttgart 1978).

According to the mode of action, but also the preferred site of use of such detergent additives, a distinction is now drawn between two generations.

The first additive generation could merely prevent the formation of deposits in the intake system, but not remove deposits already present, whereas the modern second generation additives can do both (keep-clean and clean-up effect), more particularly also due to their outstanding thermal stability in zones of relatively high temperatures, namely at the intake valves. Such detergents, which can come from a multitude of chemical substance classes, for example polyalkeneamines, polyetheramines, polybutene Mannich bases or polybutenesuccinimides, are generally employed in combination with carrier oils and in some cases further additive components, for example corrosion inhibitors and demulsifiers. The carrier oils exert a solvent or wash function in combination with the detergents. Carrier oils are generally high-boiling, viscous, thermally stable liquids which coat the hot metal surface and thus prevent the formation or deposition of impurities on the metal surface.

Recent generations of fuel additives with detergent action frequently have quaternized nitrogen groups.

For example, WO 2006/135881 describes quaternized ammonium salts, prepared by condensation of a hydrocarbyl-substituted acylating agent and of an oxygen or nitrogen atom containing compound with a tertiary amino group, and subsequent quaternization by means of hydrocarbyl epoxide in combination with stoichiometric amounts of an acid, such as more particularly acetic acid. These additives are used especially as diesel fuel additives for reducing power loss.

Polyalkene-substituted quaternized amines, such as more particularly quaternized polyisobuteneamines, and use thereof as detergent additives for reducing intake valve deposits, and as a lubricant additive for internal combustion engines, are described in US 2008/0113890.

U.S. Pat. No. 6,331,648 B1 relates to specific quaternary etheramine compounds which comprise a 1-ethyl-1,3-propylene unit incorporated between alkoxylate chain and quaternary nitrogen. There is speculation as to the usability of these compounds as anticorrosion or detergent additives in gasoline and diesel fuels, but without any demonstration of the usability thereof.

EP 182 669 A1 describes halogen- or sulfur-containing alkoxylated quaternary ammonium compounds of the general structure

[RO(R₁)ₓCH₂CH(R₂)HNR₃R₄R₆]⁺A⁻

where R₁ is an alkylene oxide block. For these compounds, a whole series of applications is postulated, including general use as fuel and lubricant additives, but without actually experimentally demonstrating specific functions. Preferred anions A− are chloride, methylsulfate and ethylsulfate.

U.S. Pat. No. 4,564,372, U.S. Pat. No. 4,581,151, U.S. Pat. No. 4,600,409 and WO 1985/000620 relate to polyoxyalkyleneamine salts quaternized, i.e. halogenated, with alkyl halides, in which polyoxyalkylene unit and amine unit via various linker groups, such as more particularly amine linker of the —C(O)—NH— type. Use as dispersants and corrosion inhibitors in fuels is postulated, but without actually experimentally demonstrating specific functions.

It is therefore an object of the present invention to provide improved quaternized fuel additives which no longer have these disadvantages of the prior art and, more particularly, are usable both in diesel fuels and gasoline fuels.

### BRIEF DESCRIPTION OF THE INVENTION

It has now been found that, surprisingly, the above object is achieved by provision of specifically additized fuels and lubricants as defined in the appended claims. The inventive additives are superior in several ways over the known prior art additives and can be used both in diesel and gasoline fuels. They are notable for their advantageous clean-up and keep-clean effect on various components of internal combustion engines, such as on diesel engine injection nozzles, but also on intake valves and injectors of gasoline engines, and prevent the formation of combustion chamber deposits or eliminate combustion chamber deposits which have already formed from internal combustion engines. They additionally prevent the formation of deposits in fuel filters or eliminate filter impurities which have already formed.

### DESCRIPTION OF FIGURES

FIG. 1 shows the injector cleanliness achievable with inventive additives after test operation with a direct injection gasoline engine (1b and 1c) compared to operation with nonadditized fuel (1a).

FIG. 2 shows the course of a one-hour engine test cycle according to CEC F-098-08.

### DETAILED DESCRIPTION OF THE INVENTION

### A1) Specific Embodiments

The present invention relates particularly to the following specific embodiments:
1. A fuel composition or lubricant composition, especially fuel composition, comprising, in a majority of a conventional fuel or lubricant, an effective amount of at least one reaction product comprising a quaternized nitrogen compound, or a component fraction thereof which is obtained from the reaction product by purification and comprises a quaternized nitrogen compound, said reaction product being obtainable by reaction
   a. of a polyether-substituted amine comprising at least one tertiary quaternizable amino group with
   b. a quaternizing agent which converts the at least one tertiary amino group to a quaternary ammonium group.
2. The fuel composition or lubricant composition according to claim 1, in which the polyether substituent comprises monomer units of the general formula Ic

—[—CH(R₃)—CH(R₄)—O—]— (Ic)
in which
R₃ and R₄ are the same or different and are each H, alkyl, alkylaryl or aryl.
3. The fuel composition or lubricant composition according to embodiment 2, wherein the polyether-substituted amine has a number-average molecular weight in the range from 500 to 5000, especially 800 to 3000 or 900 to 1500.
4. The fuel composition or lubricant composition according to any of the preceding embodiments, wherein the quaternizing agent is selected from alkylene oxides, optionally in combination with acid; aliphatic or aromatic mono- or polycarboxylic esters, such as more particularly mono- or dialkyl carboxylates; cyclic nonaromatic or aromatic mono- or polycarboxylic esters; dialkyl carbonates; alkyl sulfates; alkyl halides; alkylaryl halides; especially halogen- and sulfur-free quaternizing agents, such as alkylene oxides in combination with acid, for example a carboxylic acid; aliphatic or aromatic mono- or polycarboxylic esters, such as more particularly mono- or dialkyl carboxylates; cyclic nonaromatic or aromatic mono- or polycarboxylic esters and dialkyl carbonates; and mixtures thereof.
5. A fuel composition or lubricant composition comprising, in a majority of a conventional fuel or lubricant, an effective amount of at least one quaternized nitrogen compound of the general formula Ia or Ib

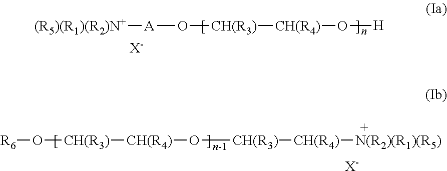

in which
R₁ and R₂ are the same or different and are each alkyl, alkenyl, hydroxyalkyl, hydroxyalkenyl, aminoalkyl or aminoalkenyl, or R₁ and R₂ together are alkylene, oxyalkylene or aminoalkylene;
R₃ and R₄ are the same or different and are each H, alkyl, alkylaryl or aryl;
R₅ is a radical introduced by quaternization, such as more particularly alkyl, hydroxyalkyl, arylalkyl or hydroxyarylalkyl;
R₆ is alkyl, alkenyl, optionally mono- or polyunsaturated cycloalkyl, aryl, in each case optionally substituted, for example by at least one hydroxyl radical or alkyl radical, or interrupted by at least one heteroatom;
A is a straight-chain or branched alkylene radical optionally interrupted by one or more heteroatoms, such as N, O and S;
n is an integer from 1 to 50 and
X⁻ is an anion, especially an anion resulting from the quaternization reaction.
6. The fuel composition or lubricant composition according to embodiment 5, in which
   R₁ and R₂ are the same or different and are each C₁-C₆-alkyl, hydroxy-C₁-C₆-alkyl, hydroxy-C₁-C₆-alkenyl, or amino-C₁-C₆-alkyl, or R₁ and R₂ together form a C₂-C₆-alkylene, C₂-C₆-oxyalkylene or C₂-C₆-aminoalkylene radical;
   R₃ and R₄ are the same or different and are each H, C₁-C₆-alkyl or phenyl;
   R₅ is a radical introduced by quaternization, selected from C₁-C₆-alkyl, hydroxy-C₁-C₆-alkyl or —CH₂CH(OH)aryl;
   R₆ is C₁-C₂₀-alkyl, for example C₁₀-C₂₀-, C₁₁-C₂₀- or C₁₂-C₂₀-alkyl, or aryl or alkylaryl, where alkyl is especially C₁-C₂₀;
   A is a straight-chain or branched C₂-C₆-alkylene radical optionally interrupted by one or more heteroatoms such as N, O and S;
   n is an integer from 1 to 30 and
   X⁻ is an anion resulting from the quaternization reaction.
7. The fuel composition according to any of the preceding embodiments, selected from diesel fuels, gasoline fuels, biodiesel fuels and alkanol-containing gasoline fuels.
8. A quaternized nitrogen compound as defined in any of the preceding embodiments, selected especially from those which are free of halogen and sulfur.
9. A process for preparing quaternized nitrogen compounds of the general formula Ia

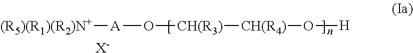

in which
R₁ to R₅, A, X and n are each as defined above
wherein
a. an aminoalkanol of the general formula II

(R₁)(R₂)N-A-OH (II)
in which
R₁, R₂ and A are each as defined above
is alkoxylated with an epoxide of the general formula III

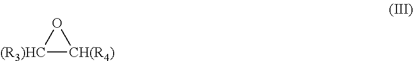

in which
R₃ and R₄ are each as defined above
to obtain an alkoxylated amine of the formula

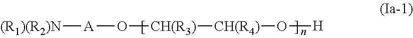

in which R₁ to R₄, A and n are each as defined above
and
b) the alkoxy compound of the formula Ia-1 thus obtained is quaternized to obtain a reaction product comprising at least one compound of the general formula Ia, the quaternization being effected, for example, with a compound of the general formula IV

R₅—X (IV)
in which
R₅ is alkyl or aryl and X is as defined above, or with an alkylene oxide of the formula

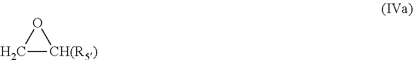

in combination with an acid HX in which X is as defined above, where R_{5′}, is H, alkyl or aryl, and the R₅ radical is a —CH₂CH(OH)R_{5′}, group.
10. A process for preparing quaternized nitrogen compounds of the general formula Ib

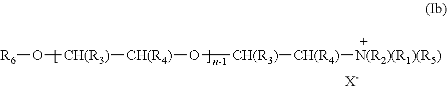

in which R₁ to R₆, X and n are each as defined above,
wherein
a) an alcohol of the general formula V

R₆—OH (V)
in which
R₆ is as defined above is alkoxylated with an epoxide of the general formula III

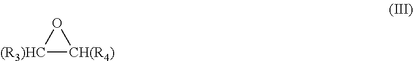

in which
R₃ and R₄ are each as defined above to obtain a polyether of the formula Ib-1;

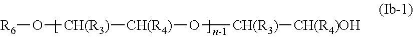

in which R₃, R₄ and R₆, A, X and n are each as defined above,
b) then the polyether of the formula Ib-1 thus obtained is aminated with an amine of the general formula

NH(R₁)(R₂) (VII)
in which R₁ and R₂ are each as defined above
to obtain an amine of the formula Ib-2

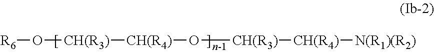

in which R₁ to R₄ and R₆, A, X and n are each as defined above,
the amine of the formula (Ib-2) is optionally alkylated if R₁ and/or R₂ is H, and then
c) the product from stage b) is quaternized to obtain a reaction product comprising at least one compound of the general formula Ib, the quaternization being effected, for example, with a compound of the general formula IV

R₅—X (IV)
in which
R₅ is alkyl or aryl and X is as defined above, or with an alkylene oxide of the formula

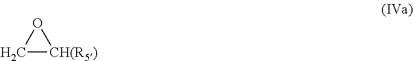

in combination with an acid HX in which X is as defined above, where R_{5′} is H, alkyl or aryl, and the R₅ radical is a —CH₂CH(OH)R_{5′} group.
11. The process according to embodiment 9 or 10, wherein the quaternizing agent is selected from: alkylene oxides, optionally in combination with an acid; alkyl carbonates, such as dialkyl carbonates; alkyl sulfates, such as dialkyl sulfates; alkyl phosphates, dialkyl phosphates, halides, such as alkyl or aryl halides; aliphatic and aromatic carboxylic esters, such as alkanoates, dicarboxylic esters; and cyclic aromatic or nonaromatic carboxylic esters.
12. A quaternized nitrogen compound obtainable by a process according to embodiment 10 or 11, especially in halogen- and sulfur-free form.
13. The use of a quaternized nitrogen compound according to embodiment 8 or prepared according to any of embodiments 9 to 11 as a fuel additive or lubricant additive.
14. The use according to embodiment 12 as a diesel fuel additive, especially as a cold flow improver or wax antisettling additive (WASA).
15. The use according to embodiment 12 as a gasoline fuel additive for reducing deposits in the intake system of a gasoline engine, such as more particularly DISI and PFI (Port Fuel Injector) engines.
16. The use according to embodiment 12 as an additive for reducing fuel consumption of direct injection diesel engines, especially of diesel engines with common rail injection systems, and/or for minimizing power loss in direct injection diesel engines, especially in diesel engines with common rail injection systems, or as an additive for reducing and/or preventing deposits in the injection systems, such as more particularly internal diesel injector deposits (IDID), and/or for reducing and/or preventing deposits in the injection nozzles in direct injection diesel engines, especially in common rail injection systems.
17. An additive concentrate comprising, in combination with further diesel or gasoline fuel additives, especially diesel fuel additives, at least one quaternized nitrogen compound as defined in embodiment 8 or prepared according to either of embodiments 9 and 10.

In a specific configuration of the invention, in some or all of the above embodiments, the quaternizing agent is not an aromatic carboxylic ester, for example a salicylic ester.

In a specific configuration of the invention, in some or all of the above embodiments, the quaternizing agent is selected from compounds of the formulae (1) and (2) described herein.

In a specific configuration of the invention, in some or all of the above embodiments, the radical (nitrogen substituent) introduced by quaternization is especially alkyl (especially C₁-C₆-alkyl) or hydroxyarylalkyl (for example 2-hydroxy-2-phenylethyl).

In a specific configuration of the invention, in some or all of the above embodiments, the polyether substituent does not have any aryl or aralkyl groups.

In a specific configuration of the invention, in some or all of the above embodiments, the quaternized nitrogen compound is a compound of the formula (Ia) or (Ib).

Test methods suitable in each case for examination of the applications referred to above are known to those skilled in the art, or are described in the experimental section which follows, to which explicit and general reference is hereby made.

### A2) General Definitions

“Halogen-free” or “sulfur-free” in the context of the present invention means the absence of inorganic or organic halogen or sulfur compounds and/or of the corresponding ions thereof, such as halide anions and sulfur-containing anions, such as more particularly sulfates. “Halogen-free” or “sulfur-free” comprises more particularly the absence of stoichiometric amounts of halogen or sulfur compounds or anions; substoichiometric amounts of halogen or sulfur compounds or anions are, for example, in molar ratios of less than 1:0.1, or less than 1:0.01 or 1:0.001, or 1:0.0001, of quaternized nitrogen compound to halogen or sulfur compound or ions thereof. “Halogen-free” or “sulfur-free” comprises, more particularly, also the complete absence of halogen or sulfur compounds and/or of the corresponding ions thereof, such as halide anion and sulfur-containing anions, such as more particularly sulfates.

“Carboxylic acids” comprise, more particularly, organic carboxylic acids, such as more particularly monocarboxylic acids of the RCOOH type in which R is a short-chain hydrocarbyl radical, for example a lower alkyl- or C₁-C₄-alkylcarboxylic acid.

“Quaternizable” nitrogen groups or amino groups comprise especially primary, secondary and tertiary amino groups.

In the absence of statements to the contrary, the following general definitions apply:

“Hydrocarbyl” should be interpreted broadly and comprises both cyclic aromatic or nonaromatic and long-chain or short-chain, straight or branched hydrocarbyl radicals having 1 to 50 carbon atoms, which may optionally additionally contain heteroatoms, for example O, N, NH, S, in the chain or ring thereof. Hydrocarbyl comprises, for example, the alkyl, alkenyl, aryl, alkylaryl, cycloalkenyl or cycloalkyl radicals defined hereinafter, and the substituted analogs thereof.

“Alkyl” or “lower alkyl” represents especially saturated, straight-chain or branched hydrocarbyl radicals having 1 to 4, 1 to 6, 1 to 8, 1 to 10, 1 to 14 or 1 to 20, carbon atoms, for example methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; and also n-heptyl, n-octyl, n-nonyl and n-decyl, n-dodecyl, n-tetradecyl, n-hexadecyl, and the singly or multiply branched analogs thereof.

“Hydroxyalkyl” represents especially the mono- or polyhydroxylated, especially monohydroxylated, analogs of the above alkyl radicals, for example the monohydroxylated analogs of the above straight-chain or branched alkyl radicals, for example the linear hydroxyalkyl groups, for example those with a primary (terminal) hydroxyl group, such as hydroxymethyl, 2-hydroxyethyl, 3-hydroxypropyl, 4-hydroxybutyl, or those with nonterminal hydroxyl groups, such as 1-hydroxyethyl, 1- or 2-hydroxypropyl, 1- or 2-hydroxybutyl or 1-, 2- or 3-hydroxybutyl.

“Alkenyl” represents mono- or polyunsaturated, especially monounsaturated, straight-chain or branched hydrocarbyl radicals having 2 to 4, 2 to 6, 2 to 8, 2 to 10 or 2 to 20 carbon atoms and a double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

“Hydroxyalkenyl” represents especially the mono- or polyhydroxylated, especially monohydroxylated, analogs of the above alkenyl radicals.

“Aminoalkyl” and “aminoalkenyl” are especially the mono- or polyaminated, especially monoaminated, analogs of the above alkyl and alkenyl radicals respectively, or analogs of the above hydroxyalkyl where the OH group is replaced by an amino group.

“Alkylene” represents straight-chain or singly or multiply branched hydrocarbylene bridging groups having 1 to 10 carbon atoms, for example C₁-C₇-alkylene groups selected from —CH₂—, —(CH₂)₂—, —(CH₂)₃—, —(CH₂)₄—, —(CH₂)₂—CH(CH₃)—, —CH₂—CH(CH₃)—CH₂—, (CH₂)₄—, —(CH₂)₅—, —(CH₂)₆, —(CH₂)₇—, —CH(CH₃)—CH₂—CH₂—CH(CH₃)— or —CH(CH₃)—CH₂—CH₂—CH₂—CH(CH₃)— or C₁-C₄-alkylene groups selected from —CH₂—, —(CH₂)₂—, —(CH₂)₃—, —(CH₂)₄—, —(CH₂)₂—CH(CH₃)—, —CH₂—CH(CH₃)—CH₂— or C₂-C₆-alkylene groups, for example —CH₂—CH(CH₃)—, —CH(CH₃)—CH₂—, —CH(CH₃)—CH(CH₃)—, —C(CH₃)₂—CH₂—, —CH₂—C(CH₃)₂—, —C(CH₃)₂—CH(CH₃)—, —CH(CH₃)—C(CH₃)₂—, —CH₂—CH(Et)-, —CH(CH₂CH₃)—CH₂—, —CH(CH₂CH₃)—CH(CH₂CH₃)—, —C(CH₂CH₃)₂—CH₂—, —CH₂—C(CH₂CH₃)₂—, —CH₂—CH(n-propyl)-, —CH(n-propyl)-CH₂—, —CH(n-propyl)-CH(CH₃)—, —CH₂—CH(n-butyl)-, —CH(n-butyl)-CH₂—, —CH(CH₃)—CH(CH₂CH₃)—, —CH(CH₃)—CH(n-propyl)-, —CH(CH₂CH₃)—CH(CH₃)—, —CH(CH₃)—CH(CH₂CH₃)—, or C₂-C₄-alkylene groups, for example selected from —(CH₂)₂—, —CH₂—CH(CH₃)—, —CH(CH₃)—CH₂—, —CH(CH₃)—CH(CH₃)—, —C(CH₃)₂—CH₂—, —CH₂—C(CH₃)₂—, —CH₂—CH(CH₂CH₃)—, —CH(CH₂CH₃)—CH₂—.

Oxyalkylene radicals correspond to the definition of the above straight-chain or singly or multiply branched alkylene radicals having 2 to 10 carbon atoms, where the carbon chain is interrupted once or more than once, especially once, by an oxygen heteroatom. Nonlimiting examples include: —CH₂—O—CH₂—, —(CH₂)₂—O—(CH₂)₂—, —(CH₂)₃—O—(CH₂)₃—, or —CH₂—O—(CH₂)₂—, —(CH₂)₂—O—(CH₂)₃—, —CH₂—O—(CH₂)₃.

“Aminoalkylene” corresponds to the definition of the above straight-chain or singly or multiply branched alkylene radicals having 2 to 10 carbon atoms, where the carbon chain is interrupted once or more than once, especially once, by a nitrogen group (especially —NH— group). Nonlimiting examples include: —CH₂—NH—CH₂—, —(CH₂)₂—NH—(CH₂)₂—, —(CH₂)₃—NH—(CH₂)₃—, or —CH₂—NH—(CH₂)₂—, —(CH₂)₂—NH—(CH₂)₃—, —CH₂—NH—(CH₂)₃.

“Alkenylene” is the mono- or polyunsaturated, especially monounsaturated, analog of the above alkylene groups having 2 to 10 carbon atoms, especially C₂-C₇-alkenylenes or C₂-C₄-alkenylene, such as —CH═CH—, —CH═CH—CH₂—, —CH₂—CH═CH—, —CH═CH—CH₂—CH₂—, —CH₂—CH═CH—CH₂—, —CH₂—CH₂—CH═CH—, —CH(CH₃)—CH═CH—, —CH₂—C(CH₃)═CH—.

“Cycloalkyl” represents carbocyclic radicals having 3 to 20 carbon atoms, for example C₃-C₁₂-cycloalkyl, such as cyclopropyl, cyclobutyl, cyclopentyl, cyclohexyl, cycloheptyl, cyclooctyl, cyclononyl, cyclodecyl, cycloundecyl and cyclododecyl; preferably cyclopentyl, cyclohexyl, cycloheptyl; and cyclopropylmethyl, cyclopropylethyl, cyclobutylmethyl, cyclobutylethyl, cyclopentyl methyl, cyclopentylethyl, cyclohexylmethyl, or C₃-C₇-cycloalkyl, such as cyclopropyl, cyclobutyl, cyclopentyl, cyclohexyl, cycloheptyl, cyclopropylmethyl, cyclopropylethyl, cyclobutylmethyl, cyclopentylethyl, cyclohexylmethyl, where the attachment to the rest of the molecule may be via any suitable carbon atom.

“Cycloalkenyl” or “mono- or polyunsaturated cycloalkyl” represents especially monocyclic mono- or polyunsaturated hydrocarbyl groups having 5 to 8 and preferably to 6 carbon ring members, for example the monounsaturated cyclopenten-1-yl, cyclopenten-3-yl, cyclohexen-1-yl, cyclohexen-3-yl and cyclohexen-4-yl radicals.

“Aryl” represents mono- or polycyclic, preferably mono- or bicyclic, optionally substituted aromatic radicals having 6 to 20, for example 6 to 10, ring carbon atoms, for example phenyl, biphenyl, naphthyl such as 1- or 2-naphthyl, tetrahydronaphthyl, fluorenyl, indenyl and phenanthrenyl. These aryl radicals may optionally bear 1, 2, 3, 4, 5 or 6 identical or different substituents.

“Alkylaryl” represents the alkyl-substituted analogs of the above aryl radicals mono- or polysubstituted, especially mono- or disubstituted, in any ring position, where aryl is likewise as defined above, for example C₁-C₄-alkylphenyl where the C₁-C₄-alkyl radicals may be in any ring position.

“Substituents” for radicals specified herein are especially selected from keto groups, —COOH, —COO-alkyl, —OH, —SH, —CN, amino, —NO₂, alkyl, or alkenyl groups.

Mn (number-average molecular weight) is determined in a conventional manner; more particularly, the figures relate to values determined by gel permeation chromatography or mass spectrometry.

### A3) Starter Compounds (Alcohols of the Formula V and Amino Alcohols of the Formula II)

### a) Alcohols of the General Formula V

R₆—OH (V)

in which R₆ is alkyl, alkenyl, optionally mono- or polyunsaturated cycloalkyl, aryl, in each case optionally substituted, for example by at least one hydroxyl radical or alkyl radical, or interrupted by at least one heteroatom;

### b) Amino Alkanols of the General Formula II

(R₁)(R₂)N-A-OH (II)

in which
R₁ and R₂ are the same or different and are each alkyl, alkenyl, hydroxyalkyl, hydroxyalkenyl, aminoalkyl or aminoalkenyl, or R₁ and R₂ together are alkylene, oxyalkylene or aminoalkylene; and
A is a straight-chain or branched alkylene or alkenylene radical optionally interrupted by one or more heteroatoms, such as N, O and S.

### A4) Quaternizing Agents

Useful quaternizing agents in principle include all compounds suitable as such. The quaternizing agent is especially selected from alkylene oxides, optionally in combination with acid; aliphatic or aromatic carboxylic esters, such as more particularly dialkyl carboxylates; alkanoates; cyclic nonaromatic or aromatic carboxylic esters; dialkyl carbonates; alkyl sulfates; alkyl halides; alkylaryl halides; and mixtures thereof.

In a particular embodiment, however, the at least one quaternizable tertiary nitrogen atom is quaternized with at least one quaternizing agent selected from epoxides, especially hydrocarbyl epoxides:

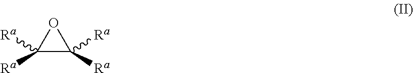

in which the R^{a} radicals present therein are the same or different and are each H or a hydrocarbyl radical. The hydrocarbyl radical may have at least 1 to 14 carbon atoms. In particular, these are aliphatic or aromatic radicals, for example linear or branched C₁-C₄-alkyl radicals, or aromatic radicals, such as phenyl or C₁-C₄-alkylphenyl.

Suitable hydrocarbyl epoxides are, for example, aliphatic and aromatic alkylene oxides, such as especially C₂₋₁₆-alkylene oxides, such as ethylene oxide, propylene oxide, 1,2-butylene oxide, 2,3-butylene oxide, 2-methyl-1,2-propene oxide (isobutene oxide), 1,2-pentene oxide, 2,3-pentene oxide, 2-methyl-1,2-butene oxide, 3-methyl-1,2-butene oxide, 1,2-hexene oxide, 2,3-hexene oxide, 3,4-hexene oxide, 2-methyl-1,2-pentene oxide, 2-ethyl-1,2-butene oxide, 3-methyl-1,2-pentene oxide, 1,2-decene oxide, 1,2-dodecene oxide or 4-methyl-1,2-pentene oxide; tetradecane oxide; hexadecene oxide; and also aromatic-substituted ethylene oxides, such as optionally substituted styrene oxide, especially styrene oxide or 4-methylstyrene oxide.

In the case of use of epoxides as quaternizing agents, they are used in the presence or in the absence of free acids, especially in the presence or absence of free protic acids, such as in particular with C₁₋₁₂-monocarboxylic acids such as formic acid, acetic acid or propionic acid, or C₂₋₁₂-dicarboxylic acids such as oxalic acid or adipic acid; or else in the presence or absence of sulfonic acids such as benzenesulfonic acid or toluenesulfonic acid, or aqueous mineral acids such as sulfuric acid or hydrochloric acid. The quaternization product thus prepared is thus either “acid-containing” or “acid-free” in the context of the present invention.

A further group of quaternizing agents includes especially alkyl esters of a cycloaromatic or cycloaliphatic mono- or polycarboxylic acid (especially of a mono- or dicarboxylic acid) or of an aliphatic polycarboxylic acid (especially dicarboxylic acid).

In a particular embodiment, the quaternization of the at least one quaternizable tertiary nitrogen atom is effected, however, with at least one quaternizing agent selected from

### a) Compounds of the General Formula 1

R₁OC(O)R₂ (1)

in which
R₁ is a lower alkyl radical and
R₂ is an optionally substituted monocyclic aryl or cycloalkyl radical, where the substituent is selected from OH, NH₂, NO₂, C(O)OR₃; R₁ₐOC(O)— in which R₁ₐ is as defined above for R₁, and R₃ is H or R₁;
or

### b) Compounds of the General Formula 2

R₁OC(O)-A-C(O)OR₁ₐ (2)

in which
R₁ and R₁ₐ are each independently a lower alkyl radical and
A is hydrocarbylene (such as alkylene or alkenylene).

Especially suitable quaternizing agents include the lower alkyl esters of oxalic acid, such as dimethyl oxalate and diethyl oxalate.

Particularly suitable compounds of the formula 1 are those in which

R₁ is a C₁-, C₂- or C₃-alkyl radical and
R₂ is a substituted phenyl radical where the substituent represents HO— or an ester radical of the formula R₁ₐOC(O)— which is in the para, meta or especially ortho position to the R₁OC(O)— radical on the aromatic ring.

Especially suitable quaternizing agents include the lower alkyl esters of salicylic acid, such as methyl salicylate, ethyl salicylate, n- and i-propyl salicylate, and n-, i- or tert-butyl salicylate.

An “anion resulting from the quaternization reaction” X⁻ is, for example, a halide, for example a chloride or bromide, a sulfate radical ((SO₄)²⁻) or the anionic radical of a mono- or polybasic, aliphatic or aromatic carboxylic acid, or the anionic radical ROC(O)O— resulting from the quaternization reaction of a dialkyl carbonate.

### A5) Quaternizable Nitrogen Compounds (of the Formula II)

The quaternizable nitrogen compound is selected from hydroxyalkyl-substituted mono- or polyamines having at least one quaternizable primary, secondary or tertiary amino group and at least one hydroxyl group which can be joined to a polyether radical.

The quaternizable nitrogen compound is especially selected from hydroxyalkyl-substituted primary, secondary, tertiary and quaternary monoamines, and hydroxyalkyl-substituted primary, secondary, tertiary and quaternary diamines.

Examples of suitable “hydroxyalkyl-substituted mono- or polyamines” are those provided with at least one hydroxyalkyl substituted, for example 1, 2, 3, 4, 5 or 6 hydroxyalkyl substituted.

Examples of “hydroxyalkyl-substituted monoamines” include: N-hydroxyalkyl monoamines, N,N-dihydroxyalkyl monoamines and N,N,N-trihydroxyalkyl monoamines, where the hydroxyalkyl groups are the same or different and are also as defined above. Hydroxyalkyl is especially 2-hydroxyethyl, 3-hydroxypropyl or 4-hydroxybutyl.

For example, the following “hydroxyalkyl-substituted polyamines” and especially “hydroxyalkyl-substituted diamines” may be mentioned: (N-hydroxyalkyl)alkylenediamines, N,N-dihydroxyalkylalkylenediamines, where the hydroxyalkyl groups are the same or different and are also as defined above. Hydroxyalkyl is especially 2-hydroxyethyl, 3-hydroxypropyl or 4-hydroxybutyl; alkylene is especially ethylene, propylene or butylene.

Mention should be made especially of the following quaternizable nitrogen compounds:

| | |
|---|---|
| NAME | FORMULA |

Alcohols with primary and secondary amine

| | |
|---|---|
| Ethanolamine | 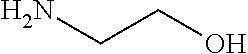 |
| 3-Hydroxy-1-propylamine | 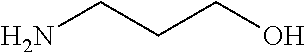 |
| Diethanolamine | 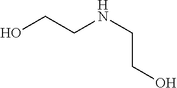 |
| Diisopropanolamine | 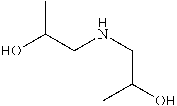 |
| N-(2-Hydroxyethyl)ethylenediamine | 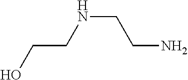 |

Alcohols with tertiary amine

| | |
|---|---|
| Triethanolamine, (2,2^{I},2^{II}-nitrilotriethanol) | 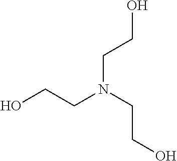 |
| 1-(3-Hydroxypropyl)imidazole | 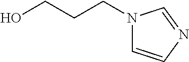 |
| Tris(hydroxymethyl)amine | 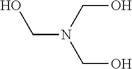 |
| 3-Dimethylamino-1-propanol | 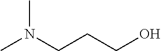 |
| 3-Diethylamino-1-propanol | 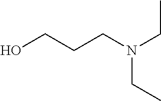 |
| 2-Diethylamino-1-ethanol | 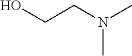 |
| 4-Diethylamino-1-butanol | 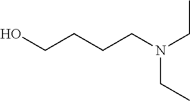 |

### A6) Preparation of Inventive Additives

### a) Preparation of the Polyether-Substituted Quaternizable Intermediates (Ia-1 and Ib-1)

a1) Proceeding from Amino Alcohols of the Formula II:

The amino alcohols of the general formula II can be alkoxylated in a manner known in principle to obtain an alkoxylated amine of the general formula Ia-1.

The performance of alkoxylations is known in principle to those skilled in the art. It is likewise known to those skilled in the art that the reaction conditions, especially the selection of the catalyst, can influence the molecular weight distribution of the alkoxylates.

For the alkoxylation, C₂-C₁₆-alkylene oxides are used, for example ethylene oxide, propylene oxide or butylene oxide. Preference is given in each case to the 1,2-alkylene oxides.

The alkoxylation may be a base-catalyzed alkoxylation. For this purpose, the amino alcohols (II) can be admixed in a pressure reactor with alkali metal hydroxides, preferably potassium hydroxide, or with alkali metal alkoxides, for example sodium methoxide. Water still present in the mixture can be drawn off by means of reduced pressure (for example <100 mbar) and/or increased temperature (30 to 150° C.). Thereafter, the alcohol is present as the corresponding alkoxide. Subsequently, inert gas (e.g. nitrogen) is used for intertization and the alkylene oxide(s) is/are added stepwise at temperatures of 60 to 180° C. up to a pressure of max. 10 bar. At the end of the reaction, the catalyst can be neutralized by adding acid (e.g. acetic acid or phosphoric acid) and can be filtered off if required. The basic catalyst can also be neutralized by adding commercial magnesium silicates, which are subsequently filtered off. Optionally, the alkoxylation can also be performed in the presence of a solvent. This may be, for example, toluene, xylene, dimethylformamide or ethylene carbonate.

The alkoxylation of the amino alcohols can also be undertaken by means of other methods, for example by acid-catalyzed alkoxylation. In addition, it is possible to use, for example, double hydroxide clays as described in DE 43 25 237 A1, or it is possible to use double metal cyanide catalysts (DMC catalysts). Suitable DMC catalysts are disclosed, for example, in DE 102 43 361 A1, especially paragraphs [0029] to [0041], and literature cited therein. For example, it is possible to use catalysts of the Zn—Co type. To perform the reaction, the amino alcohol can be admixed with the catalyst, and the mixture can be dewatered as described above and reacted with the alkylene oxides as described. Typically not more than 1000 ppm of catalyst based on the mixture are used, and due to this small amount the catalyst can remain in the product. The amount of catalyst may generally be less than 1000 ppm, for example 250 ppm or less.

The alkoxylation can alternatively also be undertaken by reaction of compounds (IV) and (V) with cyclic carbonates, for example ethylene carbonate.

a2) Proceeding from Alkanols of the Formula V:

As described in the above section a1) for amino alcohols (II), it is analogously also possible to alkoxylate alkanols R₆OH in a manner known in principle to give polyethers (Ib-1). The polyethers thus obtained can subsequently be converted to the corresponding polyetheramines (Ib-2) by reductive amination with ammonia, primary amines or secondary amines (VII) by customary methods in continuous or batchwise processes using hydrogenation or amination catalysts customary therefor, for example those comprising catalytically active constituents based on the elements Ni, Co, Cu, Fe, Pd, Pt, Ru, Rh, Re, Al, Si, Ti, Zr, Nb, Mg, Zn, Ag, Au, Os, Ir, Cr, Mo, W or combinations of these elements with one another, in customary amounts. The reaction can be performed without solvent or, in the case of high polyether viscosities, in the presence of a solvent, preferably in the presence of branched aliphatics, for example isododecane. The amine component (VII) is generally used in excess, for example in a 2- to 100-fold excess, preferably 10- to 80-fold excess. The reaction is performed at pressures of 10 to 600 bar over a period of 10 minutes to 10 hours. After cooling, the catalyst is removed by filtration, excess amine component (VII) is vaporized and the water of reaction is distilled off azeotropically or under a gentle nitrogen stream.

Should the resulting polyetheramine (Ib-2) have primary or secondary amine functionalities (R₁ and/or R₂ is H), it can subsequently be converted to a polyetheramine with tertiary amine function (R₁ and R₂ not H). The alkylation can be effected in a manner known in principle by reaction with alkylating agents. All alkylating agents are suitable in principle, for example alkyl halides, alkylaryl halides, dialkyl sulfates, alkylene oxides, optionally in combination with acid; aliphatic or aromatic carboxylic esters, such as more particularly dialkyl carboxylates; alkanoates; cyclic nonaromatic or aromatic carboxylic esters; dialkyl carbonates; and mixtures thereof. The reactions to give the tertiary polyetheramine may also take place by reductive amination, by reaction with a carbonyl compound, for example formaldehyde, in the presence of a reducing agent. Suitable reducing agents are formic acid or hydrogen in the presence of a suitable heterogeneous or homogeneous hydrogenation catalyst. The reactions can be performed without solvent or in the presence of solvents. Suitable solvents are, for example, H₂O, alkanols such as methanol or ethanol, or 2-ethylhexanol, aromatic solvents such as toluene, xylene or solvent mixtures of the Solvesso series, or aliphatic solvents, especially mixtures of branched aliphatic solvents. The reactions are performed at temperatures of 10° C. to 300° C. at pressures of 1 to 600 bar over a period of 10 minutes to 10 h. The reducing agent is used at least in a stoichiometric amount, preferably in excess, especially in a 2- to 10-fold excess.

The reaction product thus formed (polyetheramine Ib-1 or Ib-2) can theoretically be purified further, or the solvent can be removed. Usually, however, this is not absolutely necessary, such that the reaction product can be transferred without further purification into the next synthesis step, the quaternization.

### b) Quaternization

b1) With Epoxide/Acid

To perform the quaternization, the reaction product or reaction mixture from the above stage a) is admixed with at least one epoxide compound of the above formula (IVa), especially in the stoichiometric amounts required to achieve the desired quaternization. The acid is preferably likewise added in stoichiometric amounts. It is possible to use, for example, 0.1 to 2.0 equivalents, or 0.5 to 1.25 equivalents, of quaternizing agent per equivalent of quaternizable tertiary nitrogen atom. More particularly, however, approximately equimolar proportions of the epoxide are used to quaternize a tertiary amine group. Correspondingly higher use amounts are required to quaternize a secondary or primary amine group. Suitable acids are especially carboxylic acids, for example acetic acid.

Typical working temperatures here are in the range from 15 to 160° C., especially from 20 to 150 or 40 to 140° C. The reaction time may be in the range of a few minutes or a few hours, for example about 10 minutes up to about 24 hours. The reaction can be effected at a pressure of about 0.1 to 20 bar, for example 1 to 10 bar. The pressure is generally determined by the vapor pressure of the alkylene oxide used at the particular reaction temperature. More particularly, an inert gas atmosphere, for example nitrogen, is appropriate.

If required, the reactants can be initially charged for the epoxidation in a suitable organic aliphatic or aromatic solvent or a mixture thereof, or a sufficient proportion of solvent from reaction step a) is still present. Typical examples are, for example, solvents of the Solvesso series, toluene or xylene. Alkanols are additionally suitable as solvents or cosolvents in a mixture with the aforementioned solvents, for example methanol, ethanol, propanol, 2-ethylhexanol or 2-propylheptanol.

b2) With Compounds of the Formula IV

To perform the quaternization, the reaction product or reaction mixture from the above stage a) is admixed with at least one alkylating agent of the formula (IV), especially in the stoichiometric amounts required to achieve the desired quaternization. For each equivalent of quaternizable tertiary nitrogen atom, it is possible to use, for example, 0.1 to 5.0 equivalents, or 0.5 to 2.0 equivalents, of quaternizing agent. More particularly, however, approximately equimolar proportions of the alkylating agent are used to quaternize a tertiary amine group. Correspondingly higher use amounts are required to quaternize a secondary or primary amine group. Particularly suitable quaternizing agents are methyl salicylate, dimethyl oxalate, dimethyl phthalate and dimethyl carbonate.

The reaction can optionally be accelerated by adding catalytic or stoichiometric amounts of an acid. Suitable acids are, for example, proton donors such as aliphatic or aromatic carboxylic acids or fatty acids. Additionally suitable are Lewis acids, for example boron trifluoride, ZnCl₂, MgCl₂, AlCl₃ or FeCl₃. The acid can be used in amounts of 0.01 to 50% by weight, for example in the range of 0.1 to 10% by weight.

Typically, temperatures are employed here in the range from 15 to 160° C., especially from 20 to 150 or 40 to 140° C. The reaction time may be in the region of a few minutes or a few hours, for example about 10 minutes up to about 24 hours. The reaction can be effected at pressure about 0.1 to 20 bar, for example 0.5 to 10 bar. More particularly, the reaction can be effected at standard pressure. More particularly, an inert gas atmosphere, for example nitrogen, is appropriate.

If required, the reactants can be initially charged in a suitable organic aliphatic or aromatic solvent or a mixture thereof for the quaternization, or a sufficient proportion of solvent from reaction step a) is still present. Typical examples are, for example, solvents of the Solvesso series, toluene or xylene. Alkanols are additionally suitable as solvents or as cosolvents in a mixture with the aforementioned solvents, for example methanol, ethanol, propanol, butanol, 2-ethylhexanol or 2-propylheptanol.

### c) Workup of the Reaction Mixture

The reaction end product thus formed can theoretically be purified further, or the solvent can be removed. This is customary but not absolutely necessary, and so the reaction product can be used without further purification as an additive, optionally after blending with further additive components (see below). Optionally, the acid used can be removed from the reaction product by filtration, neutralization or extraction. Optionally, an excess of alkylating agent can be removed by distillation or by filtration.

### B) Further Additive Components

The fuel additized with the inventive quaternized additive is a gasoline fuel or especially a middle distillate fuel, in particular a diesel fuel.

The fuel may comprise further customary additives to improve efficacy and/or suppress wear.

In the case of diesel fuels, these are primarily customary detergent additives, carrier oils, cold flow improvers, lubricity improvers, corrosion inhibitors, demulsifiers, dehazers, antifoams, cetane number improvers, combustion improvers, antioxidants or stabilizers, antistats, metallocenes, metal deactivators, dyes and/or solvents.

In the case of gasoline fuels, these are in particular lubricity improvers (friction modifiers), corrosion inhibitors, demulsifiers, dehazers, antifoams, combustion improvers, antioxidants or stabilizers, antistats, metallocenes, metal deactivators, dyes and/or solvents.

Typical examples of suitable coadditives are listed in the following section:

### B1) Detergent Additives

The customary detergent additives are preferably amphiphilic substances which possess at least one hydrophobic hydrocarbyl radical with a number-average molecular weight (Mₙ) of 85 to 20 000 and at least one polar moiety selected from:
(Da) mono- or polyamino groups having up to 6 nitrogen atoms, at least one nitrogen atom having basic properties;
(Db) nitro groups, optionally in combination with hydroxyl groups;
(Dc) hydroxyl groups in combination with mono- or polyamino groups, at least one nitrogen atom having basic properties;
(Dd) carboxyl groups or their alkali metal or alkaline earth metal salts;
(De) sulfonic acid groups or their alkali metal or alkaline earth metal salts;
(Df) polyoxy-C₂- to C₄-alkylene moieties terminated by hydroxyl groups, mono- or polyamino groups, at least one nitrogen atom having basic properties, or by carbamate groups;
(Dg) carboxylic ester groups;
(Dh) moieties derived from succinic anhydride and having hydroxyl and/or amino and/or amido and/or imido groups; and/or
(Di) moieties obtained by Mannich reaction of substituted phenols with aldehydes and mono- or polyamines.

The hydrophobic hydrocarbyl radical in the above detergent additives, which ensures the adequate solubility in the fuel, has a number-average molecular weight (Mₐ) of 85 to 20 000, preferably of 113 to 10 000, more preferably of 300 to 5000, even more preferably of 300 to 3000, even more especially preferably of 500 to 2500 and especially of 700 to 2500, in particular of 800 to 1500. Typical hydrophobic hydrocarbyl radicals, especially in conjunction with the polar moieties, include especially polypropenyl, polybutenyl and polyisobutenyl radicals with a number-average molecular weight Mₙ of preferably in each case 300 to 5000, more preferably 300 to 3000, even more preferably 500 to 2500, even more especially preferably 700 to 2500 and especially 800 to 1500.

Examples of the above groups of detergent additives include the following:

Additives comprising mono- or polyamino groups (Da) are preferably polyalkenemono- or polyalkenepolyamines based on polypropene or on high-reactivity (i.e. having predominantly terminal double bonds) or conventional (i.e. having predominantly internal double bonds) polybutene or polyisobutene having Mₙ=300 to 5000, more preferably 500 to 2500 and especially 700 to 2500. Such additives based on high-reactivity polyisobutene, which can be prepared from the polyisobutene which may comprise up to 20% by weight of n-butene units by hydroformylation and reductive amination with ammonia, monoamines or polyamines such as dimethylaminopropylamine, ethylenediamine, diethylenetriamine, triethylenetetramine or tetraethylenepentamine, are known especially from EP-A 244 616. When polybutene or polyisobutene having predominantly internal double bonds (usually in the 13 and y positions) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or the abovementioned polyamines. Corresponding additives based on polypropene are described in particular in WO-A 94/24231.

Further particular additives comprising monoamino groups (Da) are the hydrogenation products of the reaction products of polyisobutenes having an average degree of polymerization P=5 to 100 with nitrogen oxides or mixtures of nitrogen oxides and oxygen, as described in particular in WO-A 97/03946.

Further particular additives comprising monoamino groups (Da) are the compounds obtainable from polyisobutene epoxides by reaction with amines and subsequent dehydration and reduction of the amino alcohols, as described in particular in DE-A 196 20 262.

Additives comprising nitro groups (Db), optionally in combination with hydroxyl groups, are preferably reaction products of polyisobutenes having an average degree of polymerization P=5 to 100 or 10 to 100 with nitrogen oxides or mixtures of nitrogen oxides and oxygen, as described in particular in WO-A 96/03367 and in WO-A 96/03479. These reaction products are generally mixtures of pure nitropolyisobutenes (e.g. α,β-dinitropolyisobutene) and mixed hydroxynitropolyisobutenes (e.g. α-nitro-β-hydroxypolyisobutene).

Additives comprising hydroxyl groups in combination with mono- or polyamino groups (Dc) are in particular reaction products of polyisobutene epoxides obtainable from polyisobutene having preferably predominantly terminal double bonds and Mₙ=300 to 5000, with ammonia or mono- or polyamines, as described in particular in EP-A 476 485.

Additives comprising carboxyl groups or their alkali metal or alkaline earth metal salts (Dd) are preferably copolymers of C₂- to C₄₀-olefins with maleic anhydride which have a total molar mass of 500 to 20 000 and some or all of whose carboxyl groups have been converted to the alkali metal or alkaline earth metal salts and any remainder of the carboxyl groups has been reacted with alcohols or amines. Such additives are disclosed in particular by EP-A 307 815. Such additives serve mainly to prevent valve seat wear and can, as described in WO-A 87/01126, advantageously be used in combination with customary fuel detergents such as poly(iso)buteneamines or polyetheramines.

Additives comprising sulfonic acid groups or their alkali metal or alkaline earth metal salts (De) are preferably alkali metal or alkaline earth metal salts of an alkyl sulfosuccinate, as described in particular in EP-A 639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (Df) are preferably polyethers or polyetheramines which are obtainable by reaction of C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols with 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP-A 310 875, EP-A 356 725, EP-A 700 985 and US-A 4 877 416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (Dg) are preferably esters of mono-, di- or tricarboxylic acids with long-chain alkanols or polyols, in particular those having a minimum viscosity of 2 mm²/s at 100° C., as described in particular in DE-A 38 38 918. The mono-, di- or tricarboxylic acids used may be aliphatic or aromatic acids, and particularly suitable ester alcohols or ester polyols are long-chain representatives having, for example, 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or especially imido groups (Dh) are preferably corresponding derivatives of alkyl- or alkenyl-substituted succinic anhydride and especially the corresponding derivatives of polyisobutenylsuccinic anhydride which are obtainable by reacting conventional or high-reactivity polyisobutene having Mₙ=preferably 300 to 5000, more preferably 300 to 3000, even more preferably 500 to 2500, even more especially preferably 700 to 2500 and especially 800 to 1500, with maleic anhydride by a thermal route in an ene reaction or via the chlorinated polyisobutene. The moieties having hydroxyl and/or amino and/or amido and/or imido groups are, for example, carboxylic acid groups, acid amides of monoamines, acid amides of di- or polyamines which, in addition to the amide function, also have free amine groups, succinic acid derivatives having an acid and an amide function, carboximides with monoamines, carboximides with di- or polyamines which, in addition to the imide function, also have free amine groups, or diimides which are formed by the reaction of di- or polyamines with two succinic acid derivatives. In the presence of imido moieties D(h), the further detergent additive in the context of the present invention is, however, used only up to a maximum of 100% of the weight of compounds with betaine structure. Such fuel additives are common knowledge and are described, for example, in documents (1) and (2). They are preferably the reaction products of alkyl- or alkenyl-substituted succinic acids or derivatives thereof with amines and more preferably the reaction products of polyisobutenyl-substituted succinic acids or derivatives thereof with amines. Of particular interest in this context are reaction products with aliphatic polyamines (polyalkyleneimines) such as especially ethylenediamine, diethylenetriamine, triethylenetetramine, tetraethylenepentamine, pentaethylenehexamine and hexaethyleneheptamine, which have an imide structure.

Additives comprising moieties (Di) obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or high-reactivity polyisobutene having Mₙ=300 to 5000. Such “polyisobutene Mannich bases” are described in particular in EP-A 831 141.

One or more of the detergent additives mentioned can be added to the fuel in such an amount that the dosage of these detergent additives is preferably 25 to 2500 ppm by weight, especially 75 to 1500 ppm by weight, in particular 150 to 1000 ppm by weight.

### B2) Carrier Oils

Carrier oils additionally used may be of mineral or synthetic nature. Suitable mineral carrier oils are the fractions obtained in crude oil processing, such as brightstock or base oils having viscosities, for example, from the SN 500 to 2000 class; but also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range from about 360 to 500° C., obtainable from natural mineral oil which has been catalytically hydrogenated and isomerized under high pressure and also deparaffinized). Likewise suitable are mixtures of the abovementioned mineral carrier oils.

Examples of suitable synthetic carrier oils are polyolefins (polyalphaolefins or polyinternalolefins), (poly)esters, (poly)alkoxylates, polyethers, aliphatic polyether-amines, alkylphenol-started polyethers, alkylphenol-started polyetheramines and carboxylic esters of long-chain alkanols.

Examples of suitable polyolefins are olefin polymers having Mₙ=400 to 1800, in particular based on polybutene or polyisobutene (hydrogenated or unhydrogenated).

Examples of suitable polyethers or polyetheramines are preferably compounds comprising polyoxy-C₂- to C₄-alkylene moieties which are obtainable by reacting C₂- to C₆₀-alkanols, C₆- to C₃₀-alkanediols, mono- or di-C₂- to C₃₀-alkylamines, C₁- to C₃₀-alkylcyclohexanols or C₁- to C₃₀-alkylphenols with 1 to 30 mol of ethylene oxide and/or propylene oxide and/or butylene oxide per hydroxyl group or amino group, and, in the case of the polyetheramines, by subsequent reductive amination with ammonia, monoamines or polyamines. Such products are described in particular in EP-A 310 875, EP-A 356 725, EP-A 700 985 and U.S. Pat. No. 4,877,416. For example, the polyetheramines used may be poly-C₂- to C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A 38 38 918. The mono-, di- or tricarboxylic acids used may be aliphatic or aromatic acids; suitable ester alcohols or polyols are in particular long-chain representatives having, for example, 6 to 24 carbon atoms. Typical representatives of the esters are adipates, phthalates, isophthalates, terephthalates and trimellitates of isooctanol, isononanol, isodecanol and isotridecanol, for example di(n- or isotridecyl) phthalate.

Further suitable carrier oil systems are described, for example, in DE-A 38 26 608, DE-A 41 42 241, DE-A 43 09 074, EP-A 452 328 and EP-A 548 617.

Examples of particularly suitable synthetic carrier oils are alcohol-started polyethers having about 5 to 35, preferably about 5 to 30, more preferably 10 to 30 and especially 15 to 30 C₃- to C₆-alkylene oxide units, for example selected from propylene oxide, n-butylene oxide and isobutylene oxide units, or mixtures thereof, per alcohol molecule. Nonlimiting examples of suitable starter alcohols are long-chain alkanols or phenols substituted by long-chain alkyl in which the long-chain alkyl radical is in particular a straight-chain or branched C₆- to C₁₈-alkyl radical. Particular examples include tridecanol and nonylphenol. Particularly preferred alcohol-started polyethers are the reaction products (polyetherification products) of monohydric aliphatic C₆- to C₁₈-alcohols with C₃- to C₆-alkylene oxides. Examples of monohydric aliphatic C₆-C₁₈-alcohols are hexanol, heptanol, octanol, 2-ethylhexanol, nonyl alcohol, decanol, 3-propylheptanol, undecanol, dodecanol, tridecanol, tetradecanol, pentadecanol, hexadecanol, octadecanol and the constitutional and positional isomers thereof. The alcohols can be used either in the form of the pure isomers or in the form of technical grade mixtures. A particularly preferred alcohol is tridecanol. Examples of C₃- to C₆-alkylene oxides are propylene oxide, such as 1,2-propylene oxide, butylene oxide, such as 1,2-butylene oxide, 2,3-butylene oxide, isobutylene oxide or tetrahydrofuran, pentylene oxide and hexylene oxide. Particular preference among these is given to C₃- to C₄-alkylene oxides, i.e. propylene oxide such as 1,2-propylene oxide and butylene oxide such as 1,2-butylene oxide, 2,3-butylene oxide and isobutylene oxide. Especially butylene oxide is used.

Further suitable synthetic carrier oils are alkoxylated alkylphenols, as described in DE-A 10 102 913.

Particular carrier oils are synthetic carrier oils, particular preference being given to the above-described alcohol-started polyethers.

The carrier oil or the mixture of different carrier oils is added to the fuel in an amount of preferably 1 to 1000 ppm by weight, more preferably of 10 to 500 ppm by weight and especially of 20 to 100 ppm by weight.

### B3) Cold Flow Improvers

Suitable cold flow improvers are in principle all organic compounds which are capable of improving the flow performance of middle distillate fuels or diesel fuels under cold conditions. For the intended purpose, they must have sufficient oil solubility. In particular, useful cold flow improvers for this purpose are the cold flow improvers (middle distillate flow improvers, MDFIs) typically used in the case of middle distillates of fossil origin, i.e. in the case of customary mineral diesel fuels. However, it is also possible to use organic compounds which partly or predominantly have the properties of a wax antisettling additive (WASA) when used in customary diesel fuels. They can also act partly or predominantly as nucleators. It is, though, also possible to use mixtures of organic compounds effective as MDFIs and/or effective as WASAs and/or effective as nucleators.

The cold flow improver is typically selected from:
(K1) copolymers of a C₂- to C₄₀-olefin with at least one further ethylenically unsaturated monomer;
(K2) comb polymers;
(K3) polyoxyalkylenes;
(K4) polar nitrogen compounds;
(K5) sulfocarboxylic acids or sulfonic acids or derivatives thereof; and
(K6) poly(meth)acrylic esters.

It is possible to use either mixtures of different representatives from one of the particular classes (K1) to (K6) or mixtures of representatives from different classes (K1) to (K6).

Suitable C₂- to C₄₀-olefin monomers for the copolymers of class (K1) are, for example, those having 2 to 20 and especially 2 to 10 carbon atoms, and 1 to 3 and preferably 1 or 2 carbon-carbon double bonds, especially having one carbon-carbon double bond. In the latter case, the carbon-carbon double bond may be arranged either terminally (α-olefins) or internally. However, preference is given to α-olefins, more preferably α-olefins having 2 to 6 carbon atoms, for example propene, 1-butene, 1-pentene, 1-hexene and in particular ethylene.

In the copolymers of class (K1), the at least one further ethylenically unsaturated monomer is preferably selected from alkenyl carboxylates, (meth)acrylic esters and further olefins.

When further olefins are also copolymerized, they are preferably higher in molecular weight than the abovementioned C₂- to C₄₀-olefin base monomer. When, for example, the olefin base monomer used is ethylene or propene, suitable further olefins are in particular C₁₀- to C₄₀-α-olefins. Further olefins are in most cases only additionally copolymerized when monomers with carboxylic ester functions are also used.

Suitable (meth)acrylic esters are, for example, esters of (meth)acrylic acid with C₁- to C₂₀-alkanols, especially C₁- to C₁₀-alkanols, in particular with methanol, ethanol, propanol, isopropanol, n-butanol, sec-butanol, isobutanol, tert-butanol, pentanol, hexanol, heptanol, octanol, 2-ethylhexanol, nonanol and decanol, and structural isomers thereof.

Suitable alkenyl carboxylates are, for example, C₂- to C₁₄-alkenyl esters, for example the vinyl and propenyl esters, of carboxylic acids having 2 to 21 carbon atoms, whose hydrocarbyl radical may be linear or branched. Among these, preference is given to the vinyl esters. Among the carboxylic acids with a branched hydrocarbyl radical, preference is given to those whose branch is in the α-position to the carboxyl group, the α-carbon atom more preferably being tertiary, i.e. the carboxylic acid being a so-called neocarboxylic acid. However, the hydrocarbyl radical of the carboxylic acid is preferably linear.

Examples of suitable alkenyl carboxylates are vinyl acetate, vinyl propionate, vinyl butyrate, vinyl 2-ethylhexanoate, vinyl neopentanoate, vinyl hexanoate, vinyl neononanoate, vinyl neodecanoate and the corresponding propenyl esters, preference being given to the vinyl esters. A particularly preferred alkenyl carboxylate is vinyl acetate; typical copolymers of group (K1) resulting therefrom are ethylene-vinyl acetate copolymers (“EVAs”), which are some of the most frequently used. Ethylene-vinyl acetate copolymers usable particularly advantageously and their preparation are described in WO 99/29748.

Suitable copolymers of class (K1) are also those which comprise two or more different alkenyl carboxylates in copolymerized form, which differ in the alkenyl function and/or in the carboxylic acid group. Likewise suitable are copolymers which, as well as the alkenyl carboxylate(s), comprise at least one olefin and/or at least one (meth)acrylic ester in copolymerized form.

Terpolymers of a C₂- to C₄₀-α-olefin, a C₁- to C₂₀-alkyl ester of an ethylenically unsaturated monocarboxylic acid having 3 to 15 carbon atoms and a C₂- to C₁₄-alkenyl ester of a saturated monocarboxylic acid having 2 to 21 carbon atoms are also suitable as copolymers of class (K1). Terpolymers of this kind are described in WO 2005/054314. A typical terpolymer of this kind is formed from ethylene, 2-ethylhexyl acrylate and vinyl acetate.

The at least one or the further ethylenically unsaturated monomer(s) are copolymerized in the copolymers of class (K1) in an amount of preferably 1 to 50% by weight, especially 10 to 45% by weight and in particular 20 to 40% by weight, based on the overall copolymer. The main proportion in terms of weight of the monomer units in the copolymers of class (K1) therefore originates generally from the C₂ to C₄₀ base olefins.

The copolymers of class (K1) preferably have a number-average molecular weight Mₙ of 1000 to 20 000, more preferably 1000 to 10 000 and in particular 1000 to 8000.

Typical comb polymers of component (K2) are, for example, obtainable by the copolymerization of maleic anhydride or fumaric acid with another ethylenically unsaturated monomer, for example with an α-olefin or an unsaturated ester, such as vinyl acetate, and subsequent esterification of the anhydride or acid function with an alcohol having at least 10 carbon atoms. Further suitable comb polymers are copolymers of α-olefins and esterified comonomers, for example esterified copolymers of styrene and maleic anhydride or esterified copolymers of styrene and fumaric acid. Suitable comb polymers may also be polyfumarates or polymaleates. Homo- and copolymers of vinyl ethers are also suitable comb polymers. Comb polymers suitable as components of class (K2) are, for example, also those described in WO 2004/035715 and in “Comb-Like Polymers. Structure and Properties”, N. A. Platé and V. P. Shibaev, J. Poly. Sci. Macromolecular Revs. 8, pages 117 to 253 (1974)”. Mixtures of comb polymers are also suitable.

Polyoxyalkylenes suitable as components of class (K3) are, for example, polyoxyalkylene esters, polyoxyalkylene ethers, mixed polyoxyalkylene ester/ethers and mixtures thereof. These polyoxyalkylene compounds preferably comprise at least one linear alkyl group, preferably at least two linear alkyl groups, each having 10 to 30 carbon atoms and a polyoxyalkylene group having a number-average molecular weight of up to 5000. Such polyoxyalkylene compounds are described, for example, in EP-A 061 895 and also in U.S. Pat. No. 4,491,455. Particular polyoxyalkylene compounds are based on polyethylene glycols and polypropylene glycols having a number-average molecular weight of 100 to 5000. Additionally suitable are polyoxyalkylene mono- and diesters of fatty acids having 10 to 30 carbon atoms, such as stearic acid or behenic acid.

Polar nitrogen compounds suitable as components of class (K4) may be either ionic or nonionic and preferably have at least one substituent, in particular at least two substituents, in the form of a tertiary nitrogen atom of the general formula >NR⁷ in which R⁷ is a C₈- to C₄₀-hydrocarbyl radical. The nitrogen substituents may also be quaternized, i.e. be in cationic form. An example of such nitrogen compounds is that of ammonium salts and/or amides which are obtainable by the reaction of at least one amine substituted by at least one hydrocarbyl radical with a carboxylic acid having 1 to 4 carboxyl groups or with a suitable derivative thereof. The amines preferably comprise at least one linear C₈- to C₄₀-alkyl radical. Primary amines suitable for preparing the polar nitrogen compounds mentioned are, for example, octylamine, nonylamine, decylamine, undecylamine, dodecylamine, tetradecylamine and the higher linear homologs. Secondary amines suitable for this purpose are, for example, dioctadecylamine and methylbehenylamine. Also suitable for this purpose are amine mixtures, in particular amine mixtures obtainable on the industrial scale, such as fatty amines or hydrogenated tallamines, as described, for example, in Ullmann's Encyclopedia of Industrial Chemistry, 6th Edition, “Amines, aliphatic” chapter. Acids suitable for the reaction are, for example, cyclohexane-1,2-dicarboxylic acid, cyclohexene-1,2-dicarboxylic acid, cyclopentane-1,2-dicarboxylic acid, naphthalenedicarboxylic acid, phthalic acid, isophthalic acid, terephthalic acid, and succinic acids substituted by long-chain hydrocarbyl radicals.

In particular, the component of class (K4) is an oil-soluble reaction product of poly(C₂- to C₂₀-carboxylic acids) having at least one tertiary amino group with primary or secondary amines. The poly(C₂- to C₂₀-carboxylic acids) which have at least one tertiary amino group and form the basis of this reaction product comprise preferably at least 3 carboxyl groups, especially 3 to 12 and in particular 3 to 5 carboxyl groups. The carboxylic acid units in the polycarboxylic acids have preferably 2 to 10 carbon atoms, and are especially acetic acid units. The carboxylic acid units are suitably bonded to the polycarboxylic acids, usually via one or more carbon and/or nitrogen atoms. They are preferably attached to tertiary nitrogen atoms which, in the case of a plurality of nitrogen atoms, are bonded via hydrocarbon chains.

The component of class (K4) is preferably an oil-soluble reaction product based on poly(C₂- to C₂₀-carboxylic acids) which have at least one tertiary amino group and are of the general formula IIa or IIb

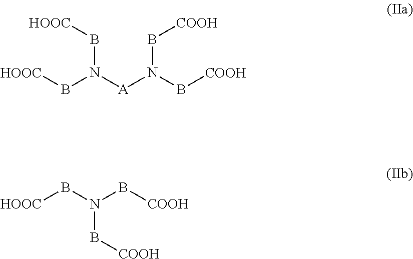

in which the variable A is a straight-chain or branched C₂- to C₆-alkylene group or the moiety of the formula III

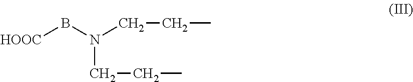

and the variable B is a C₁- to C₁₉-alkylene group. The compounds of the general formulae IIa and IIb especially have the properties of a WASA.

Moreover, the preferred oil-soluble reaction product of component (K4), especially that of the general formula IIa or IIb, is an amide, an amide-ammonium salt or an ammonium salt in which no, one or more carboxylic acid groups have been converted to amide groups.

Straight-chain or branched C₂- to C₆-alkylene groups of the variable A are, for example, 1,1-ethylene, 1,2-propylene, 1,3-propylene, 1,2-butylene, 1,3-butylene, 1,4-butylene, 2-methyl-1,3-propylene, 1,5-pentylene, 2-methyl-1,4-butylene, 2,2-dimethyl-1,3-propylene, 1,6-hexylene (hexamethylene) and in particular 1,2-ethylene. The variable A comprises preferably 2 to 4 and especially 2 or 3 carbon atoms. C₁- to C₁₉-alkylene groups of the variable B are, for example, 1,2-ethylene, 1,3-propylene, 1,4-butylene, hexamethylene, octamethylene, decamethylene, dodecamethylene, tetradecamethylene, hexadecamethylene, octadecamethylene, nonadecamethylene and especially methylene. The variable B comprises preferably 1 to 10 and especially 1 to 4 carbon atoms.

The primary and secondary amines as a reaction partner for the polycarboxylic acids to form component (K4) are typically monoamines, especially aliphatic monoamines. These primary and secondary amines may be selected from a multitude of amines which bear hydrocarbyl radicals which may optionally be bonded to one another.

These parent amines of the oil-soluble reaction products of component (K4) are usually secondary amines and have the general formula HN(R⁸)₂ in which the two variables R⁸ are each independently straight-chain or branched C₁₀- to C₃₀-alkyl radicals, especially C₁₄- to C₂₄-alkyl radicals. These relatively long-chain alkyl radicals are preferably straight-chain or only slightly branched. In general, the secondary amines mentioned, with regard to their relatively long-chain alkyl radicals, derive from naturally occurring fatty acid and from derivatives thereof. The two R⁸ radicals are preferably identical.

The secondary amines mentioned may be bonded to the polycarboxylic acids by means of amide structures or in the form of the ammonium salts; it is also possible for only a portion to be present as amide structures and another portion as ammonium salts. Preferably only few, if any, free acid groups are present. The oil-soluble reaction products of component (K4) are preferably present completely in the form of the amide structures.

Typical examples of such components (K4) are reaction products of nitrilotriacetic acid, of ethylenediaminetetraacetic acid or of propylene-1,2-diaminetetraacetic acid with in each case 0.5 to 1.5 mol per carboxyl group, especially 0.8 to 1.2 mol per carboxyl group, of dioleylamine, dipalmitinamine, dicoconut fatty amine, distearylamine, dibehenylamine or especially ditallow fatty amine. A particularly preferred component (K4) is the reaction product of 1 mol of ethylenediaminetetraacetic acid and 4 mol of hydrogenated ditallow fatty amine.

Further typical examples of component (K4) include the N,N-dialkylammonium salts of 2-N′,N′-dialkylamidobenzoates, for example the reaction product of 1 mol of phthalic anhydride and 2 mol of ditallow fatty amine, the latter being hydrogenated or unhydrogenated, and the reaction product of 1 mol of an alkenylspirobislactone with 2 mol of a dialkylamine, for example ditallow fatty amine and/or tallow fatty amine, the last two being hydrogenated or unhydrogenated.

Further typical structure types for the component of class (K4) are cyclic compounds with tertiary amino groups or condensates of long-chain primary or secondary amines with carboxylic acid-containing polymers, as described in WO 93/18115.

Sulfocarboxylic acids, sulfonic acids or derivatives thereof which are suitable as cold flow improvers of class (K5) are, for example, the oil-soluble carboxamides and carboxylic esters of ortho-sulfobenzoic acid, in which the sulfonic acid function is present as a sulfonate with alkyl-substituted ammonium cations, as described in EP-A 261 957.

Poly(meth)acrylic esters suitable as cold flow improvers of class (K6) are either homo- or copolymers of acrylic and methacrylic esters. Preference is given to copolymers of at least two different (meth)acrylic esters which differ with regard to the esterified alcohol. The copolymer optionally comprises another different olefinically unsaturated monomer in copolymerized form. The weight-average molecular weight of the polymer is preferably 50 000 to 500 000. A particularly preferred polymer is a copolymer of methacrylic acid and methacrylic esters of saturated C₁₄ and C₁₅ alcohols, the acid groups having been neutralized with hydrogenated tallamine. Suitable poly(meth)acrylic esters are described, for example, in WO 00/44857.

The cold flow improver or the mixture of different cold flow improvers is added to the middle distillate fuel or diesel fuel in a total amount of preferably 10 to 5000 ppm by weight, more preferably of 20 to 2000 ppm by weight, even more preferably of 50 to 1000 ppm by weight and especially of 100 to 700 ppm by weight, for example of 200 to 500 ppm by weight.

### B4) Lubricity Improvers

Suitable lubricity improvers or friction modifiers are based typically on fatty acids or fatty acid esters. Typical examples are tall oil fatty acid, as described, for example, in WO 98/004656, and glyceryl monooleate. The reaction products, described in U.S. Pat. No. 6,743,266 B2, of natural or synthetic oils, for example triglycerides, and alkanolamines are also suitable as such lubricity improvers.

### B5) Corrosion Inhibitors

Suitable corrosion inhibitors are, for example, succinic esters, in particular with polyols, fatty acid derivatives, for example oleic esters, oligomerized fatty acids, substituted ethanolamines, and products sold under the trade name RC 4801 (Rhein Chemie Mannheim, Germany) or HiTEC 536 (Ethyl Corporation).

### B6) Demulsifiers

Suitable demulsifiers are, for example, the alkali metal or alkaline earth metal salts of alkyl-substituted phenol- and naphthalenesulfonates and the alkali metal or alkaline earth metal salts of fatty acids, and also neutral compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example including in the form of EO/PO block copolymers, polyethyleneimines or else polysiloxanes.

### B7) Dehazers

Suitable dehazers are, for example, alkoxylated phenol-formaldehyde condensates, for example the products available under the trade names NALCO 7D07 (Nalco) and TOLAD 2683 (Petrolite).

### B8) Antifoams

Suitable antifoams are, for example, polyether-modified polysiloxanes, for example the products available under the trade names TEGOPREN 5851 (Goldschmidt), Q 25907 (Dow Corning) and RHODOSIL (Rhone Poulenc).

### B9) Cetane Number Improvers

Suitable cetane number improvers are, for example, aliphatic nitrates such as 2-ethylhexyl nitrate and cyclohexyl nitrate and peroxides such as di-tert-butyl peroxide.

### B10) Antioxidants

Suitable antioxidants are, for example substituted phenols, such as 2,6-di-tert-butylphenol and 6-di-tert-butyl-3-methylphenol, and also phenylenediamines such as N,N′-di-sec-butyl-p-phenylenediamine.

### B11) Metal Deactivators

Suitable metal deactivators are, for example, salicylic acid derivatives such as N,N′-disalicylidene-1,2-propanediamine.

### B12) Solvents

Suitable solvents are, for example, nonpolar organic solvents such as aromatic and aliphatic hydrocarbons, for example toluene, xylenes, white spirit and products sold under the trade names SHELLSOL (Royal Dutch/Shell Group) and EXXSOL (ExxonMobil), and also polar organic solvents, for example, alcohols such as 2-ethylhexanol, decanol and isotridecanol. Such solvents are usually added to the diesel fuel together with the aforementioned additives and coadditives, which they are intended to dissolve or dilute for better handling.

### C) Fuels

The inventive additive is outstandingly suitable as a fuel additive and can be used in principle in any fuels. It brings about a whole series of advantageous effects in the operation of internal combustion engines with fuels.

The present invention therefore also provides fuels, especially middle distillate fuels, with a content of the inventive quaternized additive which is effective as an additive for achieving advantageous effects in the operation of internal combustion engines, for example of diesel engines, especially of direct injection diesel engines, in particular of diesel engines with common rail injection systems. This effective content (dosage) is generally 10 to 5000 ppm by weight, preferably 20 to 1500 ppm by weight, especially 25 to 1000 ppm by weight, in particular 30 to 750 ppm by weight, based in each case on the total amount of fuel.

Middle distillate fuels such as diesel fuels or heating oils are preferably mineral oil raffinates which typically have a boiling range from 100 to 400° C. These are usually distillates having a 95% point up to 360° C. or even higher. These may also be so-called “ultra low sulfur diesel” or “city diesel”, characterized by a 95% point of, for example, not more than 345° C. and a sulfur content of not more than 0.005% by weight or by a 95% point of, for example, 285° C. and a sulfur content of not more than 0.001% by weight. In addition to the mineral middle distillate fuels or diesel fuels obtainable by refining, those obtainable by coal gasification or gas liquefaction [“gas to liquid” (GTL) fuels] or by biomass liquefaction [“biomass to liquid” (BTL) fuels] are also suitable. Also suitable are mixtures of the aforementioned middle distillate fuels or diesel fuels with renewable fuels, such as biodiesel or bioethanol.

The qualities of the heating oils and diesel fuels are laid down in detail, for example, in DIN 51603 and EN 590 (cf. also Ullmann's Encyclopedia of Industrial Chemistry, 5th edition, Volume A12, p. 617 ff.).

In addition to the use thereof in the abovementioned middle distillate fuels of fossil, vegetable or animal origin, which are essentially hydrocarbon mixtures, the inventive quaternized additive can also be used in mixtures of such middle distillates with biofuel oils (biodiesel). Such mixtures are also encompassed by the term “middle distillate fuel” in the context of the present invention. They are commercially available and usually comprise the biofuel oils in minor amounts, typically in amounts of 1 to 30% by weight, especially of 3 to 10% by weight, based on the total amount of middle distillate of fossil, vegetable or animal origin and biofuel oil.

Biofuel oils are generally based on fatty acid esters, preferably essentially on alkyl esters of fatty acids which derive from vegetable and/or animal oils and/or fats. Alkyl esters are typically understood to mean lower alkyl esters, especially C₁-C₄-alkyl esters, which are obtainable by transesterifying the glycerides which occur in vegetable and/or animal oils and/or fats, especially triglycerides, by means of lower alcohols, for example ethanol or in particular methanol (“FAME”). Typical lower alkyl esters based on vegetable and/or animal oils and/or fats, which find use as a biofuel oil or components thereof, are, for example, sunflower methyl ester, palm oil methyl ester (“PME”), soya oil methyl ester (“SME”) and especially rapeseed oil methyl ester (“RME”).

The middle distillate fuels or diesel fuels are more preferably those having a low sulfur content, i.e. having a sulfur content of less than 0.05% by weight, preferably of less than 0.02% by weight, more particularly of less than 0.005% by weight and especially of less than 0.001% by weight of sulfur.

Useful gasoline fuels include all commercial gasoline fuel compositions. One typical representative which shall be mentioned here is the Eurosuper base fuel to EN 228, which is customary on the market. In addition, gasoline fuel compositions of the specification according to WO 00/47698 are also possible fields of use for the present invention.

The inventive quaternized additive is especially suitable as a fuel additive in fuel compositions, especially in diesel fuels, for overcoming the problems outlined at the outset in direct injection diesel engines, in particular in those with common rail injection systems.

The invention is now illustrated in detail by the working examples which follow. Especially the test methods specified hereinafter form part of the general disclosure of the application and are not limited to the specific working examples.

### EXPERIMENTAL SECTION

### A. General Test Methods

### 1. XUD9 Test—Determination of Flow Restriction

The procedure was according to the standard stipulations of CEC F-23-1-01.

### 2. DW10 Test—Determination of Power Loss as a Result of Injector Deposits in the Common Rail Diesel Engine

### 2.1. DW10-KC—Keep-Clean Test

The keep-clean test is based on CEC test procedure F-098-08 Issue 5. This is done using the same test setup and engine type (PEUGEOT DW10) as in the CEC procedure.

### Change and Special Features:

In the tests, cleaned injectors were used. The cleaning time in the ultrasound bath in water+10% Superdecontamine (Intersciences, Brussels) at 60° C. was 4 h.

### Test Run Times:

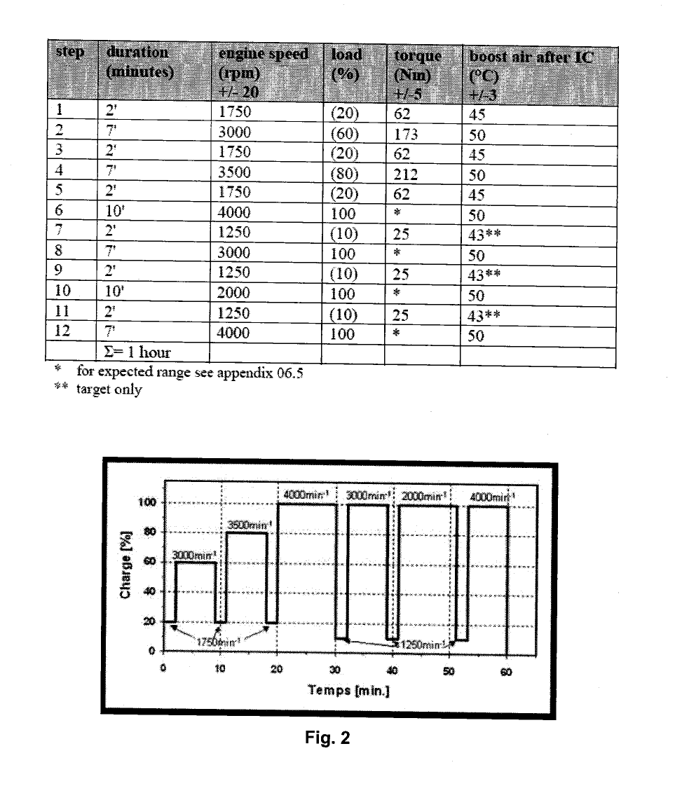

The test run time was 12 h without shutdown phases. The one-hour test cycle from CEC F-098-08, shown in FIG. 2, was run through 12 times.

### Performance Determination:

The initial power P0,KC [kW] is calculated from the measured torque at full load 4000/min directly after the test has started and the engine has run hot. The procedure is described in Issue 5 of the test procedure (CEC F-98-08). This is done using the same test setup and the PEUGEOT DW10 engine type.

The final performance (Pend,KC) is determined in the 12th cycle in stage 12 (see table, FIG. 2). Here too, the operation point is full load 4000/min. Pend,KC [kW] is calculated from the torque measured.

The power loss in the KC test is calculated as follows:

$$Power loss , KC [ \% ] = ( 1 - \frac{Pend , KC}{P 0 , KC} ) * 100$$

### 2.2. DW10 Dirty-Up Clean-Up (DU-CU)

The DU-CU test is based on CEC test procedure F-098-08 Issue 5. The procedure is described in Issue 5 of the test procedure (CEC F-98-08). This is done using the same test setup and the PEUGEOT DW10 engine type.

The DU-CU test consists of two individual tests which are run in succession. The first test serves to form deposits (DU), the second to remove the deposits (CU). After the DU, the power loss is determined. After the end of the DU run, the engine is not operated for at least 8 hours and is cooled to ambient temperature. Thereafter, the CU fuel is used to start the CU without deinstalling and cleaning the injectors. The deposits and power loss ideally decline over the course of the CU test.

### Change and Special Features:

Cleaned injectors were installed in the engine prior to each DU test. The cleaning time in the ultrasound bath at 60° C., in water+10% Superdecontamine (Intersciences, Brussels), was 4 h.

### Test Run Times:

The test run time was 12 h for the DU and 12 h for the CU. The engine was operated in the DU and CU tests without shutdown phases.

The one-hour test cycle from CEC F-098-08, shown in FIG. 2, was run through 12 times in each case.

### Performance Determination:

The initial power P0,du [kW] is calculated from the measured torque at full load 4000/min directly after the test has started and the engine has run hot. The procedure is likewise described in Issue 5 of the test procedure.

The final performance (Pend,du) is determined in the 12th cycle in stage 12 (see table above). Here too, the operation point is full load 4000/min. Pend,du [kW] is calculated from the torque measured.

The power loss in the DU is calculated as follows:

$$Power loss , du [ \% ] = ( 1 - \frac{Pend , du}{P 0 , du} ) * 100$$

### Clean-Up

The initial power P0,cu [kW] is calculated from the measured torque at full load 4000/min directly after the test has started and the engine has run hot in the CU. The procedure is likewise described in Issue 5 of the test procedure.

The final performance (Pend,cu) is determined in the 12th cycle in stage 12 (see table, FIG. 2). Here too, the operation point is full load 4000/min. Pend,cu [kW] is calculated from the torque measured.

The power loss in the CU test is calculated as follows (negative number for the power loss in the cu test means an increase in performance)

$$Power loss ( DU , CU ) [ \% ] = ( \frac{Pend , du - pend , cu}{P 0 , du} ) * 100$$

The fuel used was a commercial diesel fuel from Haltermann (RF-06-03). To artificially induce the formation of deposits at the injectors, 1 ppm by weight of zinc in the form of a zinc didodecanoate solution was added thereto.

### 3. IDID Test—Determination of Additive Effect on Internal Injector Deposits

The formation of deposits within the injector was characterized by the deviations in the exhaust gas temperatures of the cylinders at the cylinder outlet on cold starting of the DW10 engine.

To promote the formation of deposits, 1 mg/l of sodium salt of an organic acid, 20 mg/l of dodecenylsuccinic acid and 10 mg/l of water were added to the fuel.

The test is conducted as a dirty-up clean-up test (DU-CU).

DU-CU is based on CEC test procedure F-098-08 Issue 5.

The DU-CU test consists of two individual tests which are run in succession. The first test serves to form deposits (DU), the second to remove the deposits (CU).

After the DU run, after a rest phase of at least eight hours, a cold start of the engine is conducted, followed by idling for 10 minutes.

Thereafter, the CU fuel is used to start the CU without deinstalling and cleaning the injectors. After the CU run over 8 h, after a rest phase of at least eight hours, a cold start of the engine is conducted, followed by idling for 10 minutes. The evaluation is effected by the comparison of the temperature profiles for the individual cylinders after the cold start in the du and CU runs.

The IDID test indicates the formation of internal deposits in the injector. The characteristic used in this test is the exhaust gas temperature of the individual cylinders. In an injector system without IDIDs, the exhaust gas temperatures of the cylinders increase homogeneously. In the presence of IDIDs, the exhaust gas temperatures of the individual cylinders do not increase homogeneously and deviate from one another.

The temperature sensors are beyond the cylinder head outlet in the exhaust gas manifold. Significant deviation of the individual cylinder temperatures (e.g. >20° C.) indicates the presence of internal injector deposits (IDIDs).

The tests (DU and CU) are each conducted with run time 8 h. The one-hour test cycle from CEC F-098-08 is run through 8 times in each case. In the event of deviations of the individual cylinder temperatures of greater than 45° C. from the mean for all 4 cylinders, the test is stopped early.

### B. Preparation and Analysis Examples

### Reactants Used:

| | | |
|---|---|---|
| N,N-dimethylethanolamine | CAS. 108-01-0 | from BASF |
| 1,2-Propylene oxide | CAS. 75-56-9 | from BASF |
| 1,2-Butylene oxide | CAS. 106-88-7 | from BASF |
| Potassium tert-butoxide | CAS. 865-47-4 | from Aldrich |
| Dimethyl oxalate | CAS. 553-90-2 | from Aldrich |
| Solvent Naphtha Heavy | CAS. 64742-94-5 | from Exxon Mobil |
| Styrene oxide | CAS. 96-09-3 | from Aldrich |
| 2-Ethylhexanol | CAS. 104-76-7 | from BASF |
| Lauric acid | CAS. 143-07-7 | from Aldrich |
| Isotridecanol N | CAS. 27458-92-0 | from BASF |
| Acetic acid, pure | CAS. 64-19-7 | from Aldrich |
| Formic acid, 85% in H₂O | CAS 64-18-6 | from Kraft |
| Formalin, 36.5% | CAS 50-00-0 | from Aldrich |

Polydispersities D were determined by means of gel permeation chromatography.

### Synthesis Example 1

### N,N-Dimethylethanolamine*15 PO (A)

In a 2 l autoclave, N,N-dimethylethanolamine (76.7 g) is admixed with potassium tert-butoxide (4.1 g). The autoclave is purged three times with N₂, a supply pressure of approx. 1.3 bar of N₂ is established and the temperature is increased to 130° C. 1,2-Propylene oxide (750 g) is metered in over a period of 10 h, in such a way that the temperature remains between 129° C.-131° C. This is followed by stirring at 130° C. for 6 h, purging with N₂, cooling to 60° C. and emptying of the reactor. Excess propylene oxide is removed under reduced pressure on a rotary evaporator. The basic crude product is neutralized with the aid of commercial magnesium silicates, which are subsequently filtered off. This gives 831 g of the product in the form of an orange oil (TBN 58.1 mg KOH/g; D 1.16).

### Synthesis Example 2

### N,N-Dimethylethanolamine*25 BuO (B)

In a 2 l autoclave, N,N-dimethylethanolamine (47.1 g) is admixed with potassium tert-butoxide (5.0 g). The autoclave is purged three times with N₂, a supply pressure of approx. 1.3 bar of N₂ is established and the temperature is increased to 140° C. 1,2-Butylene oxide (953 g) is metered in over a period of 9 h, in such a way that the temperature remains between 138° C.-141° C. This is followed by stirring at 140° C. for 6 h, purging with N₂, cooling to 60° C. and emptying of the reactor. Excess butylene oxide is removed under reduced pressure on a rotary evaporator. The basic crude product is neutralized with the aid of commercial magnesium silicates, which are subsequently filtered off. This gives 1000 g of the product in the form of a yellow oil (TBN 28.1 mg KOH/g; D 1.12).

### Synthesis Example 3

### N,N-Dimethylethanolamine*15 PO Quaternized with dimethyl oxalate (I)

Polyetheramine (A) (250 g) from Synthesis example 1 is admixed with dimethyl oxalate (59 g) and lauric acid (12.5 g) and the reaction mixture is stirred at a temperature of 120° C. for 4 h. Subsequently, excess dimethyl oxalate is removed at a temperature of 120° C. on a rotary evaporator under reduced pressure (p=5 mbar). This gives 290 g of the product. ¹H NMR analysis of the quaternized polyetheramine thus obtained shows the quaternization.

### Synthesis Example 4

### N,N-Dimethylethanolamine*25 BuO Quaternized with dimethyl oxalate (II)

Polyetheramine (B) (250 g) from Synthesis example 2 is admixed with dimethyl oxalate (67.3 g) and lauric acid (6.2 g) and the reaction mixture is stirred at a temperature of 120° C. for 4.5 h. Subsequently, excess dimethyl oxalate is removed at a temperature of 120° C. on a rotary evaporator under reduced pressure (p=5 mbar). This gives 270 g of the product. ¹H NMR analysis of the quaternized polyetheramine thus obtained shows the quaternization.

### Synthesis Example 5

### N,N-Dimethylethanolamine*25 BuO Quaternized with styrene oxide/acetic acid (III)

Polyetheramine (B) (400 g) from Synthesis example 2 is dissolved in Solvent Naphtha Heavy (436 g), admixed with styrene oxide (24.0 g) and acetic acid (12.0 g), and then stirred at a temperature of 80° C. for 8 h. After cooling to room temperature, 870 g of the product are obtained. ¹H NMR analysis of the solution of the quaternized polyetheramine in Solvent Naphtha Heavy thus obtained shows the quaternization.

### Synthesis Example 6

### N,N-Dimethylethanolamine*15 PO Quaternized with propylene oxide/acetic acid (IV)

In a 2 l autoclave, polyetheramine (A) (305 g) from Synthesis example 1 is dissolved in 2-ethylhexanol (341 g) and admixed with acetic acid (18.3 g). The autoclave is purged three times with N₂, a supply pressure of approx. 1.3 bar of N₂ is established and the temperature is increased to 130° C. 1,2-Propylene oxide (17.7 g) is metered in. This is followed by stirring at 130° C. for 5 h, purging with N₂, cooling to 40° C. and emptying of the reactor. Excess propylene oxide is removed on a rotary evaporator under reduced pressure. This gives 675 g of the product in the form of an orange oil. ¹H NMR analysis of the solution of the quaternized polyetheramine in 2-ethylhexanol thus obtained shows the quaternization.

### Synthesis Example 7

### N,N-Dimethylethanolamine*15 PO Quaternized with ethylene oxide/acetic acid (V)

In a 2 l autoclave, polyetheramine (A) (518 g) from Synthesis example 1 is dissolved in 2-ethylhexanol (570 g) and admixed with conc. acetic acid (30 g). The autoclave is purged three times with N₂, a supply pressure of approx. 1.3 bar of N₂ is established and the temperature is increased to 130° C. Ethylene oxide (22 g) is metered in. This is followed by stirring at 130° C. for 5 h, purging with N₂, cooling to 40° C. and emptying of the reactor. This gives 1116 g of the product in the form of an orange oil. ¹H NMR analysis of the solution of the quaternized polyetheramine in 2-ethylhexanol thus obtained shows the quaternization.

### Synthesis Example 8

### Isotridecanol N*22 BuO: polyether (C)

The polyether is prepared from isotridecanol N and 1,2-butylene oxide in a molar ratio of 1:22 according to known processes, by DMC catalysis as described, for example, in EP1591466A.

### Synthesis Example 9

### Isotridecanol (Tridecanol N, BASF)*22 BuO Aminated with NH₃: prim. polyetheramine (D)

The prim. polyetheramine (D) is prepared by reaction of the polyether (C) from Synthesis example 8 with NH₃ in the presence of a suitable hydrogenation catalyst according to known processes, as described, for example, in DE3826608A. The analysis of the polyetheramine (D) thus obtained gives TBN 32.0 mg KOH/g.

### Synthesis Example 10

### tert-Polyetheramine (E)

The polyetheramine (D) (400 g) from Synthesis example 9 is admixed with formic acid (65.3 g, 85% in H₂O) while cooling with an ice bath. The reaction mixture is subsequently warmed up to a temperature of 45° C., and formaldehyde solution (44.9 g, 36.5% in H₂O) is added dropwise at this temperature, in the course of which the carbon dioxide released is drawn off from the reaction vessel. The reaction mixture is stirred at a temperature of 80° C. for 16 h. Subsequently, the reaction mixture is cooled to room temperature, admixed with hydrochloric acid (37%; 35.4 g) and stirred at room temperature for 1 h. H₂O (500 ml) is added and the aqueous phase is adjusted to a pH of approx. 10 by adding 50% potassium hydroxide solution. Subsequently, the mixture is extracted repeatedly with tert-butyl methyl ether (1200 ml in total). The combined organic phases are washed with sat. aqueous NaCl solution and dried over MgSO₄, and the solvent is removed under reduced pressure. This gives 403 g of the product in the form of a yellow oil. ¹H NMR analysis of the tert-polyetheramine thus obtained shows the reductive dimethylation.

### Synthesis Example 11

### Isotridecanol (Tridecanol N, BASF)*22 BuO Aminated with NH₃, red. dimethylated, Quaternized with dimethyl oxalate (VI)

tert-Polyetheramine (E) (172 g) from Synthesis example 10 is admixed with dimethyl oxalate (55.3 g) and lauric acid (5.2 g), and the reaction mixture is stirred at a temperature of 120° C. for 4 h. Subsequently, excess dimethyl oxalate is removed on a rotary evaporator under reduced pressure (p=5 mbar) at a temperature of 120° C. ¹H NMR analysis of the quaternized polyetheramine thus obtained shows the quaternization.

### Synthesis Example 12

### Isotridecanol (Tridecanol N, BASF)*22 BuO Aminated with NH₃, dimethylated, Quaternized styrene oxide/acetic acid (VII)

tert-Polyetheramine (E) (200 g) from Synthesis example 10 is dissolved in toluene (222 g), admixed with styrene oxide (14.4 g) and conc. acetic acid (7.2 g), and then stirred at a temperature of 80° C. for 7 h. ¹H NMR analysis of the solution thus obtained shows the quaternization.

### C. Use Examples

In the use examples which follow, the additives are used either as a pure substance (as synthesized in the above preparation examples) or in the form of an additive package.

### Use Example 1

### Determination of the Additive Action on the Formation of Deposits in Diesel Engine Injection Nozzles

### a) XUD9 Tests

Fuel used: RF-06-03 (reference diesel, Haltermann Products, Hamburg)

The results are compiled in Table 1.

TABLE 1
XUD9 tests

| | | Dosage according to | Flow restriction |
|---|---|---|---|
| | | preparation example | 0.1 mm needle |
| | | [mg of active | stroke |
| Ex. | Designation | ingredient/kg] | [%] |
| #1 | M1, according to | 30 | 20.9 |
| | preparation example 4 | | |
| #2 | M2, according to | 30 | 44.5 |
| | preparation example 3 | | |

### b) DW10 Test

The test results are shown in Table 2.

TABLE 2
Results of the DW10 tests

| | Dose | Power loss | Power loss | Power loss |
|---|---|---|---|---|
| Additive | [mg/kg] | KC | DU | DU-CU |
| Base value | 0 | 5.0% | | |
| M1, according to preparation | 80 | 0.61% | | |
| example 6, keep-clean | | | | |
| M2, according to preparation | 75 | | 2.8% | 1.7% |
| example 3, clean-up | | | | |

### Use Example 2

### Intake Valve Cleanliness (Gasoline Engine with Suction Tube Injection)

Method: MB M102 E (CEC F-05-93)

Fuel: E5 according to EN 228

Additive according to Synthesis example 4

### Results:

| | Intake valve deposits after end of test (mg/V) |
|---|---|
| Base value (no additive) | 112 |
| With 116 mg/kg of additive | 86 |

### Use Example 3

### Injector Cleanliness

### (Direct Injection Gasoline Engine)

Method: BASF in-house method

Engine: turbocharged four-cylinder of capacity 1.6 liters

Test duration: 60 hours

Fuel: test fuel with 7% by volume of oxygen-containing components

Additives:

A: additive according to Synthesis example 4

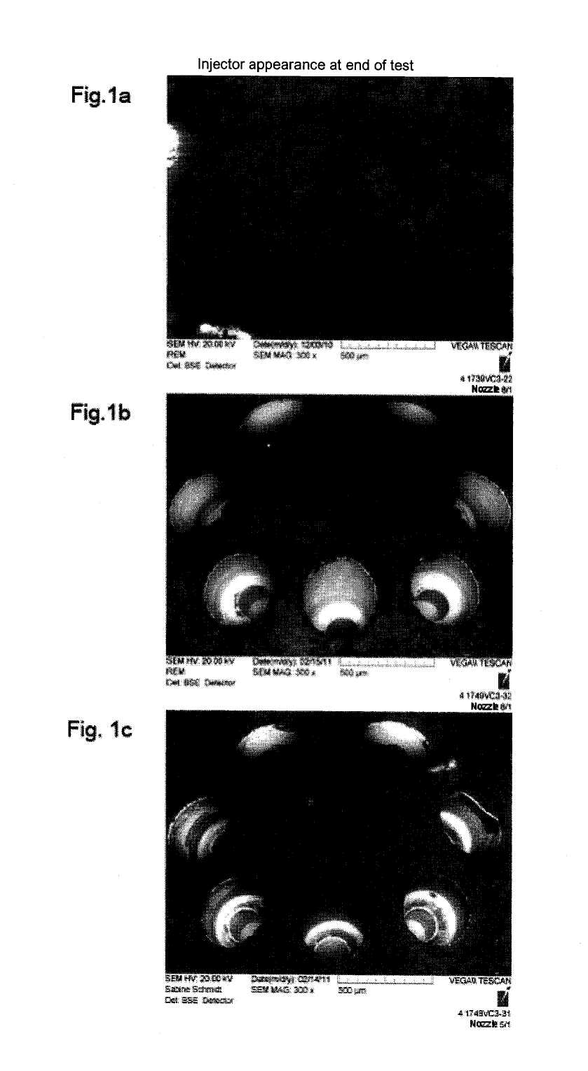

B: additive according to Synthesis example 3

| | FR* | FR* | |
|---|---|---|---|
| | Start of test | End of test | Injector appearance at end of test |
| Base value (no | 0.95 | 1.00 | see FIG. 1a |
| additive) | | | |
| With 116 mg/kg | 0.96 | 0.94 | see FIG. 1b |
| of additive A | | | |
| With 116 mg/kg | 0.96 | 0.93 | see FIG. 1c |
| of additive B | | | |

*The FR is a parameter detected by the engine control, which correlates with the duration of the injection operation of the fuel into the combustion chamber. The more marked is the formation of deposits in the injector nozzles, the longer the injection time and higher the FR. Conversely, the FR remains constant or tends to decrease slightly when the injector nozzles remain free of deposits.

Reference is made explicitly to the disclosure of the publications cited herein.

1. A fuel composition or lubricant composition comprising, in a conventional fuel or lubricant, at least one reaction product comprising a quaternized nitrogen compound, said reaction product being obtainable by reaction
a) of a polyether-substituted amine comprising at least one tertiary quaternizable amino group with
b) a quaternizing agent which converts the at least one tertiary amino group to a quaternary ammonium group.

2. The fuel composition or lubricant composition according to claim 1, in which the polyether substituent comprises monomer units of the general formula Ic
—[—CH(R₃)—CH(R₄)—O—]— (Ic)
in which
R₃ and R₄ are the same or different and are each H, alkyl, alkylaryl or aryl.

3. The fuel composition or lubricant composition according to claim 2, wherein the polyether-substituted amine has a number-average molecular weight in the range from 500 to 5000.

4. The fuel composition or lubricant composition according to any of the preceding claims, wherein the quaternizing agent is selected from alkylene oxides, optionally in combination with acid; aliphatic or aromatic carboxylic esters, such as more particularly dialkyl carboxylates; alkanoates; cyclic nonaromatic or aromatic carboxylic esters; alkyl sulfates; alkyl halides; alkylaryl halides; and mixtures thereof.

5. A fuel composition or lubricant composition comprising, in a majority of a conventional fuel or lubricant, an effective amount of at least one quaternized nitrogen compound of the general formula Ia or Ib 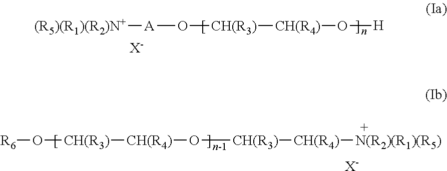
in which
R₁ and R₂ are the same or different and are each alkyl, alkenyl, hydroxyalkyl, hydroxyalkenyl, aminoalkyl or aminoalkenyl, or R₁ and R₂ together are alkylene, oxyalkylene or aminoalkylene;
R₃ and R₄ are the same or different and are each H, alkyl, alkylaryl or aryl;
R₅ is a radical introduced by quaternization, such as more particularly alkyl, hydroxyalkyl, arylalkyl or hydroxyarylalkyl;
R₆ is alkyl, alkenyl, optionally mono- or polyunsaturated cycloalkyl, aryl, in each case optionally substituted, for example by at least one hydroxyl radical or alkyl radical, or interrupted by at least one heteroatom;
A is a straight-chain or branched alkylene radical optionally interrupted by one or more heteroatoms, such as N, O and S;
n is an integer from 1 to 50 and
X⁻ is an anion, especially an anion resulting from the quaternization reaction.

6. The fuel composition or lubricant composition according to claim 5, in which R₁ and R₂ are the same or different and are each C₁-C₆-alkyl, hydroxy-C₁-C₆-alkyl, hydroxy-C₁-C₆-alkenyl, or amino-C₁-C₆-alkyl, or R₁ and R₂ together form a C₂-C₆-alkylene, C₂-C₆-oxyalkylene or C₂-C₆-aminoalkylene radical;
R₃ and R₄ are the same or different and are each H, C₁-C₆-alkyl or phenyl;
R₅ is a radical introduced by quaternization, selected from C₁-C₆-alkyl, hydroxy-C₁-C₆-alkyl or —CH₂CH(OH)aryl;
R₆ is C₁-C₂₀-alkyl or aryl or alkylaryl;
A is a straight-chain or branched C₂-C₆-alkylene radical optionally interrupted by one or more heteroatoms such as N, O and S;
n is an integer from 1 to 30 and
X⁻ is an anion resulting from the quaternization reaction.

7. The fuel composition according to any of the preceding claims, selected from diesel fuels, gasoline fuels, biodiesel fuels and alkanol-containing gasoline fuels.

8. A quaternized nitrogen compound as defined in any of the preceding claims.

9. A process for preparing quaternized nitrogen compounds of the general formula Ia 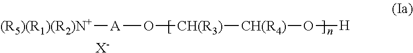
in which
R₁ to R₅, A, X and n are each as defined above,
wherein
a) an aminoalkanol of the general formula II
(R₁)(R₂)N-A-OH (II)
in which
R₁, R₂ and A are each as defined above
is alkoxylated with an epoxide of the general formula III 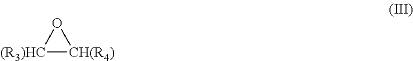
in which
R₃ and R₄ are each as defined above
to obtain an alkoxylated amine of the formula 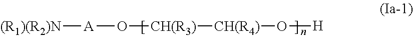
in which R₁ to R₄, A and n are each as defined above
and
b) the alkoxy compound of the formula Ia-1 thus obtained is quaternized to obtain a reaction product comprising at least one compound of the general formula Ia, the quaternization being effected, for example, with a compound of the general formula IV
R₅—X (IV)
in which
R₅ is alkyl or aryl and X is as defined above, or with an alkylene oxide of the formula 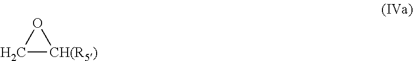
in combination with an acid HX in which X is as defined above, where R_{5′} is H, alkyl or aryl, and the R₅ radical is a —CH₂CH(OH)R_{5′} group.

10. A process for preparing quaternized nitrogen compounds of the general formula Ib 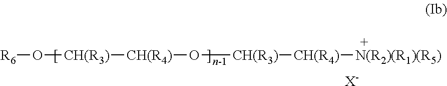
in which R₁ to R₆, X and n are each as defined above,
wherein
a) an alcohol of the general formula V
R₆—OH (V)
in which
R₆ is as defined above is alkoxylated with an epoxide of the general formula III 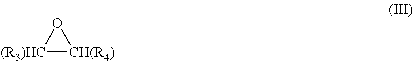
in which
R₃ and R₄ are each as defined above to obtain a polyether of the formula Ib-1; 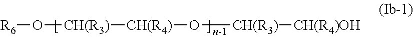
in which R₃, R₄ and R₆, A, X and n are each as defined above,
b) then the polyether of the formula Ib-1 thus obtained is aminated with an amine of the general formula
NH(R₁)(R₂) (VII)
in which R₁ and R₂ are each as defined above
to obtain an amine of the formula Ib-2 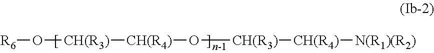
in which R₁ to R₄ and R₆, A, X and n are each as defined above,
the amine of the formula (Ib-2) is optionally alkylated if R₁ and/or R₂ is H, and then
c) the product from stage b) is quaternized to obtain a reaction product comprising at least one compound of the general formula Ib, the quaternization being effected, for example, with a compound of the general formula IV
R₅—X (IV)
in which
R₅ is alkyl or aryl and X is as defined above, or with an alkylene oxide of the formula 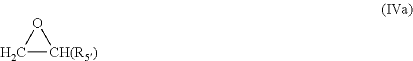
in combination with an acid HX in which X is as defined above, where R_{5′} is H, alkyl or aryl, and the R₅ radical is a —CH₂CH(OH)R_{5′} group.

11. The process according to claim 9 or 10, wherein the quaternizing agent is selected from: alkylene oxides, optionally in combination with an acid; alkyl carbonates, such as dialkyl carbonates; alkyl sulfates, such as dialkyl sulfates; alkyl phosphates, dialkyl phosphates, halides, such as alkyl or aryl halides; aliphatic and aromatic carboxylic esters, such as alkanoates, dicarboxylic esters; and cyclic aromatic or nonaromatic carboxylic esters.

12. A quaternized nitrogen compound obtainable by a process according to claim 10 or 11.

13. The use of a quaternized nitrogen compound according to claim 8 or prepared according to any of claims 9 to 11 as a fuel additive or lubricant additive.

14. The use according to claim 13 as a diesel fuel additive, especially as a cold flow improver or wax antisettling additive (WASA).

15. The use according to claim 13 as a gasoline fuel additive for reducing or preventing deposits in the intake system of a gasoline engine, especially for reducing or preventing deposits in injection nozzles of direct injection gasoline engines.

16. The use according to claim 13 as an additive for reducing fuel consumption of direct injection diesel engines, especially for reducing the fuel consumption of diesel engines with common rail injection systems, and/or for minimizing power loss in direct injection diesel engines, especially in diesel engines with common rail injection systems, or as an additive for reducing and/or preventing deposits in the injection systems, such as more particularly internal diesel injector deposits (IDID), and/or for reducing and/or preventing deposits in the injection nozzles in direct injection diesel engines, especially in common rail injection systems.

17. An additive concentrate comprising, in combination with further diesel or gasoline fuel additives, at least one quaternized nitrogen compound as defined in claim 8 or prepared according to either of claims 9 and 10.
